(19) United States
(12) Patent Application Publication
(10) Pub. No.: US 2022/0027018 A1
(43) Pub. Date: Jan. 27, 2022

(54) MONITOR, AND METHOD FOR COMBINED DISPLAY OF PHYSIOLOGICAL SIGN PARAMETERS AND MEDICATION INFORMATION FOR SAME

(71) Applicant: SHENZHEN MINDRAY BIO-MEDICAL ELECTRONICS CO., LTD., Shenzhen (CN)

(72) Inventors: Lin TAN, Shenzhen (CN); Ruiling PAN, Shenzhen (CN); Jian CEN, Shenzhen (CN); Xiaoling ZOU, Shenzhen (CN); Weiwei YUAN, Shenzhen (CN)

(73) Assignee: SHENZHEN MINDRAY BIO-MEDICAL ELECTRONICS CO., LTD., Shenzhen (CN)

(21) Appl. No.: 17/492,607

(22) Filed: Oct. 2, 2021

Related U.S. Application Data
(63) Continuation-in-part of application No. PCT/CN2019/080995, filed on Apr. 2, 2019.
Application No. PCT/CN2019/080995 is a continuation-in-part of application No. PCT/CN2019/080935, filed on Apr. 2, 2019.

(51) Int. Cl. G06F 3/0481; G06F 3/0484; G06T 11/20; A61M 5/168

(52) U.S. Cl. CPC G06F 3/0481; G06F 3/0484; G06T 11/206; A61M 5/16877; A61M 5/16886; G06T 2210/41; A61M 2205/3569; A61M 2205/3389; A61M 2205/583; A61M 2205/581; A61M 2205/584; A61M 2205/3303; A61M 2205/502

(57) ABSTRACT

This disclosure provides a monitor, and a method performed by the monitor for displaying one or more physiological sign parameters and medication information in association. The monitor includes a physiological data monitoring device, a processor, a display, and a communication unit. The physiological data monitoring device is used for monitoring the one or more physiological sign parameters of a monitored object, and the communication unit is used for receiving the medication information from an infusion pump. The processor is used for controlling the display screen for displaying the physiological sign parameters and the medication information in association on the monitor.

### CROSS-REFERENCE TO RELATED APPLICATIONS

This application is a bypass continuation-in-part of Patent Cooperation Treaty Application No. PCT/CN2019/080995, filed on Apr. 2, 2019, and also a bypass continuation-in-part of Patent Cooperation Treaty Application No. PCT/CN2019/080935, filed on Apr. 2, 2019. Both applications are hereby incorporated by reference in their entireties.

### TECHNICAL FIELD

The disclosure relates to the field of medical monitoring, and in particular to a monitor, and a method for combined display of physiological sign parameters and medication information for same.

### BACKGROUND

During the course of a patient's medication, there are many drugs that affect changes of the patient's physiological sign parameters. Under the action of efficacy of drugs, some changes in vital signs are rapid and remarkable, while other changes are gradual. A physician evaluates the effect of the drugs based on the changes in the patient's physiological parameters, then determines whether the medication strategy needs to be adjusted, and then adopts a more appropriate treatment strategy.

However, a current monitoring system and an infusion system work independently of each other, and information of the monitoring system and the infusion system is not well integrated for use. Consequently, medical staff can obtain related information only from respective outputs of the two systems, and then think about combining the information of the two systems by themselves, so as to analyze a relationship between medication and changes in the patient's physiological parameters. Such an association is extremely implicit and inaccurate. In many clinical drug research projects, researchers have to create a table by themselves to record drug information along with physiological parameter values and then draw a graph to facilitate analysis of a change trend of the patient's physiological parameters under the action of drugs.

### SUMMARY

The disclosure provides a monitor, and a method for combined display of physiological sign parameters and medication information for same, so as to solve the above technical problem.

An embodiment of the disclosure provides a monitor including a physiological data monitoring apparatus, a processor, a display, and a communication unit, where the physiological data monitoring apparatus is configured to monitor physiological sign parameter(s) of a monitored object, the communication unit is configured to receive medication information from an infusion pump, and the processor is configured to control the display to display the physiological sign parameter(s) and the medication information in association.

An embodiment of the disclosure further provides a method for combined display of physiological sign parameter(s) and medication information, where the method is applied to a monitor and includes the following steps: monitoring physiological sign parameter(s) of a monitored object; receiving medication information from an infusion pump; and performing associated display of the physiological sign parameter(s) and the medication information on the monitor.

In another aspect, this disclosure provides a method for displaying physiological sign parameter, which includes: detecting a medical and nursing operation on a monitored object, and determining an occurrence time of the medical and nursing operation; determining a target physiological sign parameter associated with the medical and nursing operation; recording multiple parameter information of the target physiological sign parameter of the monitored object from the occurrence time; and displaying the multiple parameter information.

In an embodiment, determining an occurrence time of the medical and nursing operation includes: receiving the occurrence time that is input by a user for the medical and nursing operation; or determining an operation device for the medical and nursing operation, and acquiring the occurrence time of the medical and nursing operation from the operation device.

In an embodiment, determining a target physiological sign parameter associated with the medical and nursing operation includes: receiving a physiological sign parameter type that is input by a user for the medical and nursing operation as the target physiological sign parameter; or determining, according to a preset correspondence between medical and nursing operations and physiological sign parameters, the target physiological sign parameter corresponding to the detected medical and nursing operation; or obtaining parameter information of an optional physiological sign parameter within a preset time period, and determining the optional physiological sign parameter, which has a change trend of the parameter information meeting a preset condition, as the target physiological sign parameter.

In an embodiment, recording multiple parameter information of the target physiological sign parameter of the monitored object from the occurrence time includes: keeping recording the parameter information of the target physiological sign parameter of the monitored object from the occurrence time until receiving a recording stop instruction.

In an embodiment, recording multiple parameter information of the target physiological sign parameter of the monitored object from the occurrence time includes: keeping recording, from the occurrence time, the parameter information of the target physiological sign parameter of the monitored object within the preset time period.

In an embodiment, displaying the multiple parameter information includes: generating a trend chart and/or a waveform chart corresponding to the medical and nursing operation according to the multiple parameter information; and displaying the trend chart and/or the waveform chart corresponding to the medical and nursing operation.

In an embodiment, displaying the trend chart and/or the waveform chart corresponding to the medical and nursing operation includes: switching a review trend chart and/or a review waveform chart displayed currently to the trend chart and/or the waveform chart corresponding to the medical and nursing operation.

In an embodiment, displaying the trend chart and/or the waveform chart corresponding to the medical and nursing operation includes: displaying the trend chart and/or the waveform chart corresponding to the medical and nursing operation concurrently together with displaying the review trend chart and/or the review waveform chart.

In an embodiment, displaying the trend chart and/or the waveform chart corresponding to the medical and nursing operation concurrently on together with displaying the review trend chart and/or the review waveform chart includes: marking the trend chart and/or the waveform chart corresponding to the medical and nursing operation on the displayed review trend chart and/or the review waveform chart.

In an embodiment, displaying the trend chart and/or the waveform chart corresponding to the medical and nursing operation concurrently on together with displaying the review trend chart and/or the review waveform chart includes: dividing a trend chart region in a physiological sign parameter interface into a first sub-region and a second sub-region according to a preset region proportion; displaying the review trend chart and/or the review waveform chart in the first sub-region; and displaying the trend chart and/or the waveform chart corresponding to the medical and nursing operation in the second sub-region.

In an embodiment, displaying the trend chart and/or the waveform chart corresponding to the medical and nursing operation concurrently together with displaying the review trend chart and/or the review waveform chart includes: using two vertically overlapped layers in a trend chart region of a physiological sign parameter interface to respectively display the review trend chart and/or the review waveform chart and the trend chart and/or the waveform chart corresponding to the medical and nursing operation.

In an embodiment, a display pattern of the review trend chart and/or the review waveform chart is different from a display pattern of the trend chart and/or the waveform chart corresponding to the medical and nursing operation.

In an embodiment, displaying the trend chart and/or the waveform chart corresponding to the medical and nursing operation concurrently together with displaying the review trend chart and/or the review waveform chart includes: popping up a floating interface at an associated position in a trend chart region in a physiological sign parameter interface, wherein the trend chart region is configured to display the review trend chart and/or the review waveform chart; and displaying, in the floating interface, the trend chart and/or the waveform chart corresponding to the medical and nursing operation.

In an embodiment, the physiological sign parameter display method further includes: determining an alarm condition corresponding to the target physiological sign parameter; and determining the parameter information that meets the alarm condition in the displayed multiple parameter information, and prompting the parameter information that meets the alarm condition.

In an embodiment, the parameter information is a parameter value; the target physiological sign parameter is provided with a preset target parameter value, which is used for indicating a target situation that the parameter value of the target physiological sign parameter is desired to reach after the medical and nursing operation is performed on the monitored object; and the alarm condition is that a proportion of a difference value between the parameter value of the target physiological sign parameter and the target parameter value to the target parameter value is higher than or lower than a preset proportion value.

In an embodiment, the physiological sign parameter display method further includes: determining an alarm condition corresponding to the target physiological sign parameter; and marking for prompting a parameter value that meets the alarm condition in the trend chart and/or the waveform chart corresponding to the medical and nursing operation.

In an embodiment, marking for prompting of a parameter value that meets the alarm condition in the trend chart and/or the waveform chart corresponding to the medical and nursing operation includes: determining the parameter value that meets the alarm condition in the trend chart and/or the waveform chart corresponding to the medical and nursing operation; and when multiple parameter values meet the alarm condition, marking for prompting maximum and minimum values of the multiple parameter values in the trend chart and/or the waveform chart corresponding to the medical and nursing operation.

In an embodiment, the physiological sign parameter display method further includes: displaying preset detailed information associated with the parameter value in response to a triggering operation on a marked parameter value by a user.

In an embodiment, the target physiological sign parameter is provided with a preset target parameter value, which is used for indicating a target situation that the parameter value of the target physiological sign parameter is desired to reach after the medical and nursing operation is performed on the monitored object; and the alarm condition is that a proportion of a difference value between the parameter value of the target physiological sign parameter and the target parameter value to the target parameter value is higher than or lower than a preset proportion value.

In an embodiment, marking for prompting a parameter value that meets the alarm condition includes: determining an alarm type of the parameter value that meets the alarm condition, wherein the alarm type comprises a high alarm type and a low alarm type, the high alarm type is used for indicating that the proportion is higher than the preset proportion value and the parameter value is greater than the target parameter value, and the low alarm type is used for indicating that the proportion is higher than the preset proportion value and the parameter value is less than the target parameter value; determining an alarm prompt corresponding to the alarm type; and displaying the alarm prompt at an associated position of the parameter value that meets the alarm condition.

In an embodiment, marking for prompting a parameter value that meets the alarm condition further includes: displaying the preset proportion value at the associated position of the alarm prompt.

In an embodiment, the physiological sign parameter display method further includes: prompting the target parameter value corresponding to the target physiological sign parameter in the trend chart and/or the waveform chart corresponding to the medical and nursing operation.

In an embodiment, prompting the target parameter value corresponding to the target physiological sign parameter includes: displaying the target parameter value and/or a mark line corresponding to the target parameter value in the trend chart and/or the waveform chart corresponding to the medical and nursing operation.

In an embodiment, the physiological sign parameter display method further includes: when the mark line corresponding to the target parameter value is displayed, determining a line that does not coincide with the mark line in the trend chart and/or the waveform chart corresponding to the medical and nursing operation; and filling a region, formed by the line and the mark line, with a background color.

In an embodiment, the physiological sign parameter display method further includes: obtaining a real-time parameter value of the target physiological sign parameter of the monitored object, and obtaining a target parameter value corresponding to the target physiological sign parameter, wherein the target parameter value is used for indicating a target situation that the parameter value of the target physiological sign parameter is desired to reach after the medical and nursing operation is performed on the monitored object; and displaying the real-time parameter value and the target parameter value.

In an embodiment, the physiological sign parameter display method further includes: comparing the real-time parameter value and the target parameter value to obtain a comparison result; and prompting the comparison result.

In an embodiment, prompting the comparison result includes: determining a content prompt of the comparison result, and displaying the content prompt.

In an embodiment, the physiological sign parameter display method further includes: obtaining a real-time parameter value of the target physiological sign parameter of the monitored object, and obtaining an alarm condition corresponding to the target physiological sign parameter, wherein the target physiological sign parameter is provided with a preset target parameter value, which is used for indicating a target situation that the parameter value of the target physiological sign parameter is desired to reach after the medical and nursing operation is performed on the monitored object; and the alarm condition is that a proportion of a difference value between the parameter value of the target physiological sign parameter and the target parameter value to the target parameter value is higher than or lower than a preset proportion value of the target parameter value; determining an alarm type according to the real-time parameter value and the alarm condition; and prompting the alarm type.

In an embodiment, prompting the alarm type includes: determining a content prompt of the alarm type, and displaying the content prompt.

In an embodiment, the medical and nursing operation comprises medication operation related operations; and the method further includes: obtaining the medication operation related information, the related information comprising one or more of an occurrence time of medication operation, a drug name, a total drug volume, a drug flow rate, and used drug volume; and displaying the medication operation related information.

In an embodiment, the medication related operation includes regulating the drug flow rate; and displaying the medication operation related information includes: generating a line chart including a flow rate before regulation and a flow rate after regulation according to the regulation of the drug flow rate; and displaying the line chart.

In an embodiment, displaying the medication operation related information includes: when there are multiple drugs corresponding to the medication operation, displaying the respective used drug volume of each drug; or when there are multiple drugs corresponding to the medication operation, displaying a total volume of the used drug volumes of all the drugs.

In an embodiment, displaying the medication operation related information includes: determining a preset medication region in a physiological sign parameter interface, with a number of drug related information that can be displayable in the preset medication region being a preset number; when the number of the drug related information corresponding to the medication operation is greater than the preset number, displaying the preset number of the drug related information in the preset medication region; and displaying, in response to a switching operation by a user, in the preset medication region, a remaining number of the drug related information that are not displayed.

In an embodiment, displaying the multiple parameter information includes:

determining a parameter information region in a physiological sign parameter interface, with a number of a target physiological sign parameter that can be displayable in the parameter information region being a preset number; when the number of the target physiological sign parameters is greater than the preset number, displaying the preset number of the parameter information of the target physiological sign parameters in the parameter information region; and displaying, in response to the switching operation by a user, in the parameter information region, a remaining number of the parameter information of the target physiological sign parameters that are not displayed.

In another aspect, this disclosure provides a medical device, including: a display configured to display information; a memory storing executable program instructions; and a processor, which executes the executable program instructions to implement steps of the physiological sign parameter display method described above.

In yet another aspect, this disclosure further provides a readable storage medium with a computer program stored therein, where steps of the physiological sign parameter display method described above are implemented when the computer program is executed by a processor.

According to the monitor, and the method for combined display of the physiological sign parameter(s) and medication information for same, the physiological sign parameter(s) of the monitored object and the medication information from the infusion pump are combined for associated display on the monitor to reflect the impact of medication on the physiological sign parameter(s), thereby facilitating analysis of the effects of drugs and bedside medication administration.

### BRIEF DESCRIPTION OF THE DRAWINGS

In order to more clearly describe the technical solutions in the embodiments of the disclosure or in the prior art, the drawings required for describing the embodiments or the prior art are briefly described below. Apparently, the drawings in the following description show only some of the embodiments of the disclosure, and those of ordinary skill in the art may still derive other drawings from these drawings without creative efforts.

FIG. 1 is a schematic diagram of modules of a monitor and an infusion pump connected over a network according to an embodiment of the disclosure;

FIG. 2 is a schematic diagram showing combined display of physiological sign parameters and medication information of an infusion pump by a monitor according to an embodiment of the disclosure;

FIG. 3 is a diagram of state examples for a pump status according to an embodiment of the disclosure;

FIG. 4 is a schematic diagram of combined display of physiological sign parameters and medication information that are associated based on time according to an embodiment of the disclosure;

FIG. 5 is a schematic diagram of a watch window for assisting an operator in determining whether a medication administration flow rate is appropriate according to an embodiment of the disclosure;

FIG. 6 is a schematic diagram of combined display of physiological sign parameters and medication information that are associated based on distribution according to an embodiment of the disclosure;

FIG. 7 is a max-min-average trend chart according to an embodiment of the disclosure;

FIG. 8 is a principle diagram of removing an abnormal value according to an embodiment of the disclosure;

FIG. 9 is an example diagram of a statistical result of a liquid infusion volume according to an embodiment of the disclosure;

FIG. 10 is a complete example diagram of combined display of physiological sign parameters and medication information of an infusion pump according to an embodiment of the disclosure;

FIG. 11 is a schematic flowchart of a method for combined display of physiological sign parameters and medication information of an infusion pump according to an embodiment of the disclosure;

FIG. 12 is a schematic flow diagram for a method for displaying physiological sign parameter;

FIGS. 13A-13J are multiple example views of a display interface; and

FIG. 14 is a structural schematic diagram of a monitor.

### DETAILED DESCRIPTIONS

The technical solutions of the embodiments of the disclosure are described below clearly and comprehensively in conjunction with accompanying drawings of the embodiments of the disclosure. Apparently, the embodiments described are only some of, rather than all of, the embodiments of the disclosure. Based on the embodiments in the disclosure, all other embodiments derived by those of ordinary skill in the art without creative efforts shall all fall within the scope of protection of the disclosure.

It can be understood that the terms in the description and the claims of the disclosure as well as the above accompanying drawings are only used to describe specific embodiments, and are not intended to limit the disclosure. The terms “first”, “second”, etc. in the description and the claims of the disclosure as well as the above accompanying drawings are used to distinguish different objects, rather than to describe a specific order. Singular forms “a/an” and “the” are intended to include a plural form unless stated explicitly otherwise. The terms “including” and any variations thereof are intended to cover non-exclusive inclusion. For example, a process, a method, a system, a product, or a device that includes a series of steps or units is not limited to the listed steps or units, but optionally further includes unlisted steps or units, or optionally further includes other steps or units inherent in these processes, methods, products, or devices. In addition, the disclosure may be implemented in various forms and is not limited to the embodiments described in the embodiments.

Preferred embodiments of the disclosure are subsequently described in the description, but the description is for the purpose of explaining the general principles of the disclosure and is not intended to limit the scope of the disclosure. The scope of protection of the disclosure is defined by the appended claims.

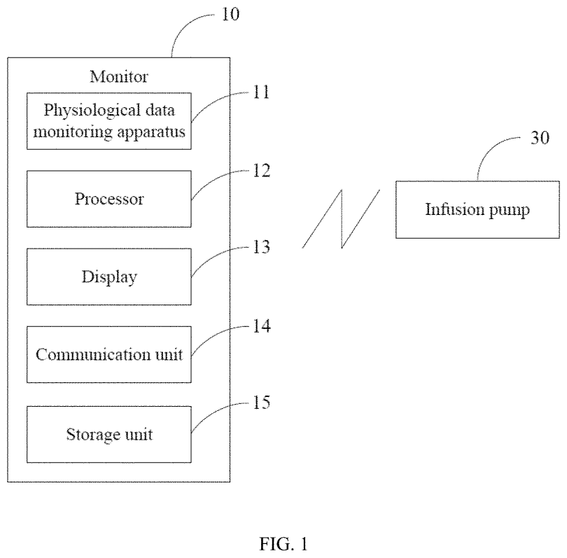

Referring to FIG. 1, FIG. 1 is a schematic diagram of modules of a monitor 10 and an infusion pump 30 connected over a network according to an embodiment of the disclosure. The monitor 10 includes a physiological data monitoring apparatus 11, a processor 12, a display 13, and a communication unit 14. The processor 12 establishes a communication connection to each of the physiological data monitoring apparatus 11, the display 13, and the communication unit 14. Specifically, the physiological data monitoring apparatus 11 is an apparatus for monitoring a heart rate, a respiration rate, blood oxygen, blood pressure, body temperature, etc. of a monitored object. The processor 12 may be a central processing unit (CPU) or another general-purpose processor, a digital signal processor (DSP), an application-specific integrated circuit (ASIC), a field-programmable gate array (FPGA), or another programmable logic device, a discrete gate or transistor logic device, a discrete hardware component, etc. The general-purpose processor may be a microprocessor, or the general-purpose processor may be any conventional processor, etc. The display 13 is various screens that can be used for information display, for example, an LCD screen. The communication unit 14 may be, but is not limited to, a wired communication device and a wireless communication device, where the wireless communication device may be Bluetooth, near-field communication, wireless fidelity (Wi-Fi), and a telemetry antenna, for example, a wireless medical telemetry service (WMTS) antenna.

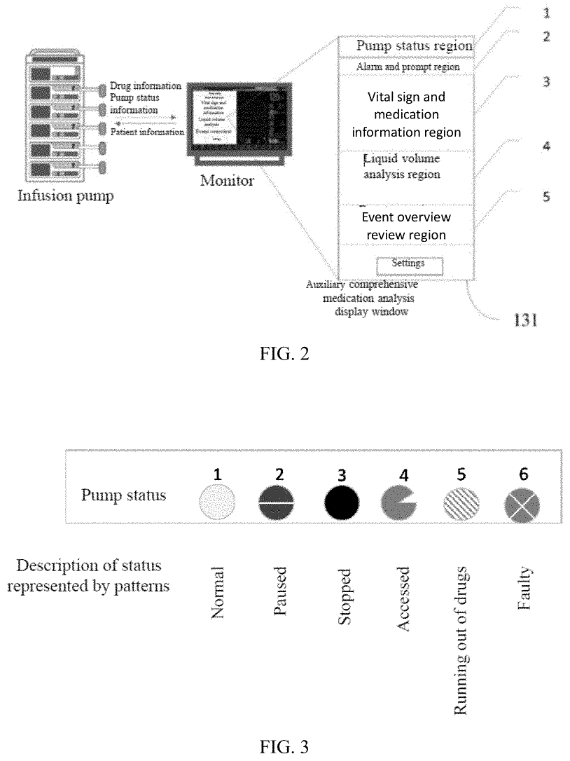

Referring also to FIG. 2, FIG. 2 is a schematic diagram showing combined display of physiological sign parameters and medication information of an infusion pump by a monitor according to an embodiment of the disclosure. The physiological data monitoring apparatus 11 is configured to monitor the physiological sign parameters of a monitored object. The communication unit 14 is configured to receive the medication information from the infusion pump 30. The processor 12 is configured to control the display 13 to perform associated display of the physiological sign parameters and the medication information.

The medication information includes at least one of a flow rate of a drug, a dose, a concentration, a cumulative infusion liquid volume, and a physiological sign parameter that is mainly affected during the use of the drug, where the physiological sign parameter that is mainly affected during the use of the drug is at least one of the monitored physiological sign parameters.

Specifically, in one embodiment, the monitored physiological sign parameters of the monitored object include, but are not limited to, a heart rate, a respiration rate, blood oxygen, blood pressure, and body temperature. The infusion pump 30 includes, but is not limited to, a transfusion pump and a syringe pump. The infusion pump 30 establishes a network connection to the monitor 10 in a wired or wireless manner. In this way, the infusion pump 30 sends pump status information and the medication information to the monitor 10. The communication unit 14 of the monitor 10 receives the pump status information and the medication information. The pump status information includes, but is not limited to: a pump start/pause/stop state, a normal or exceptional state, and a pump's access door open/closed state. The medication information includes, but is not limited to: a pump number, a drug name, a drug label color, a flow rate, a dose, a concentration, a remaining liquid volume, a cumulative infusion liquid volume, a working mode, and a physiological sign parameter that is mainly affected (for example, blood pressure mainly affected by vasopressor drugs). When receiving the pump status information and the medication information from the infusion pump 30, the processor 12 at least controls the display 13 to perform associated display of the physiological sign parameter that is mainly affected during the use of the drug and the medication information.

Therefore, the processor 12 of the monitor 10 can control the display 13 to perform the associated display of the physiological sign parameters of the monitored object and the medication information, such that an association between the physiological sign parameters and the medication information is presented in a more comprehensible form, to help physicians analyze an association between a medication administration process and fluctuation of a vital sign parameter, and also make it convenient for nurses to perform bedside medication administration.

It can be understood that, in one embodiment, when the monitor 10 and the infusion pump 30 establish a wired or wireless network connection, the infusion pump 30 can also obtain monitored object information from the monitor 10. The monitored object information includes, but is not limited to: a patient ID number, a medical record number, a bed number, name, gender, height, weight, age, etc. Therefore, the monitored object information and the medication information can be associated and stored in the infusion pump 30.

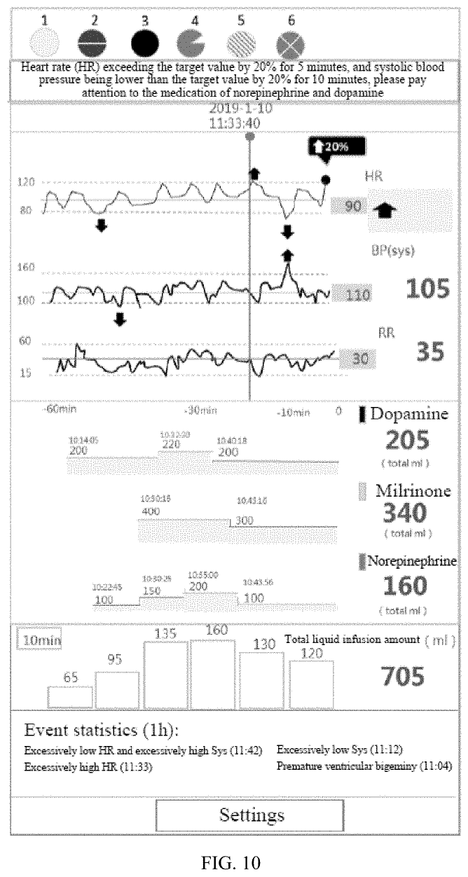

Specifically, the processor 12 controls the display 13 to display an auxiliary comprehensive medication analysis display window 131. The auxiliary comprehensive medication analysis display window 131 includes at least a pump status region 1, an alarm and prompt region 2, a vital sign and medication information display region 3, a liquid volume analysis region 4, and an event overview region 5. FIG. 10 is a complete example diagram of associated display of physiological sign parameters and medication information of an infusion pump according to an embodiment of the disclosure. This is described in detail below.

### Pump Status Region 1

In one embodiment, the processor 12 controls the display 13 to display received information about a pump status of each pump of the infusion pump 30 in the pump status region 1, and the pump status specifically includes, but is not limited to: started, paused, stopped, accessed, running out of drugs, and faulty. Each pump state is indicated by a corresponding shape, color, and/or character, which specifically includes, but is not limited to, different pump states indicated by graphic transformation, color changes, or text changes. For example, in one embodiment, as shown in FIG. 3, a pump state of a pump 1 is normal in operation; a pump state of a pump 2 is paused in infusion, in which case an operator may be dealing with air bubbles in a liquid path and temporarily stops medication administration; a pump state of a pump 3 is stopped, indicating that this pump no longer performs medication administration; a pump state of a pump 4 is accessed, indicating that an access door of this pump is opened; a pump state of a pump 5 is running out of drugs, which reminds the operator to replenish drugs in time or prepare to end the infusion; and a pump state of a pump 6 is faulty, indicating that a current fault of the pump 6 needs to be dealt with in time, otherwise it affects the normal medication administration. It can be understood that in other embodiment variants, a pump state of each pump can also be indicated by a combination of one or more of graphic transformation, color transformation, text transformation, graphic blinking, light blinking, and sound transformation. Especially in the pump state of running out of drugs or in the faulty state, a status alarm is issued by a combination of one or more of graphic transformation, color transformation, text transformation, graphic blinking, light blinking, and sound transformation, so as to more effectively remind the operator to deal with it.

Further, the monitor 10 further includes a storage unit 15. The storage unit 15 is electrically connected to the processor 12. The storage unit 15 may include a high-speed random access memory, and may further include a non-volatile memory such as a hard disk, an internal memory, and a removable hard disk, a smart media card (SMC), a secure digital (SD) card, a flash card, a plurality of disk storage devices, a flash memory device, or another volatile solid-state storage device. When the display 13 displays the pump status information and the status alarm, the processor 12 further associates the pump status information and the status alarm with the monitored object information and controls the storage unit 15 to store association data of the pump status information, the status alarm, and the monitored object information in patient data to serve as event reference information for querying the patient data.

### Alarm and Prompt Region 2

The processor 12 is further configured to, when unusual fluctuation occurs in any physiological sign parameter monitored by the physiological data monitoring apparatus 11, control the display 13 to mainly present, in the alarm and prompt region 2, prompt information that is related to a medication effect. In one embodiment, it is controlled to generate the prompt information when unusual fluctuation occurs in any physiological sign parameter monitored by the physiological data monitoring apparatus 11. The physiological sign parameter may be a physiological sign parameter closely related to medication information, or may be a physiological sign parameter that is less related or not related to the medication information such that when unusual fluctuation occurs, the operator is reminded to pay attention to control over a drug usage amount or an injection flow rate closely related to the vital sign parameter.

It can be understood that, in one embodiment, the prompt information includes not only prompt information, but also alarm information. The alarm may be, but is not limited to, a sound alarm signal and a light alarm signal, or may be a voice prompt signal or display signal with corresponding content, etc.

Specifically, in one embodiment, when the use of one or more drugs is related to one or more physiological sign parameters of the monitored object, it is desired in clinical treatment to stabilize measured values of the physiological sign parameters within a reasonable target range. It can be understood that the operator can define the target range as a target range suitable for the monitored object based on a physiological condition of the monitored object. When the measured values of the physiological sign parameters of the patient fluctuate within the target range, it indicates that the fluctuation of the physiological sign parameters is normal; when the measured values of the physiological sign parameters of the monitored object exceed the target range, it indicates that unusual fluctuation occurs in the physiological sign parameters, and the processor 12 controls to generate prompt information.

In one embodiment, the content of the prompt information includes a combination of one or more of a specific pattern, color, text, voice, and video, that is, the prompt information can be characterized by a specific pattern, color, text, voice, or video. Therefore, the operator can quickly understand the content of the prompt information, which makes it convenient for the operator to perform a next step in time.

It can be understood that, in other embodiments, the processor 12 also controls to issue prompt information when the measured values of other physiological sign parameters of the monitored object that are not associated with or have little association with the use of drug exceed a target range corresponding to the measured values of the physiological sign parameters.

Further, in one embodiment, the storage unit 15 stores a predefined correspondence between a fluctuation range of each physiological sign parameter and a prompt level. The processor 12 determines and controls to generate prompt information corresponding to the prompt level according to the correspondence and a current range of unusual fluctuation of the physiological sign parameter. For example, the level may be, but is not limited to, high, medium, low, etc. Therefore, the operator can tell a current prompt level, for example, high, medium, and low, according to current prompt information in a working process, and give priority to a monitored object with a high prompt level.

For example, in one embodiment, a vasoactive drug, norepinephrine, is administered to a monitored object with hypotension, and norepinephrine increases blood pressure of the monitored object. Clinically, for the monitored object with hypotension, most operators give a blood pressure target range according to the condition of the monitored object, for example, maintaining blood pressure within 20 mmHg fluctuation at 120 mmHg/85 mmHg (systolic/diastolic blood pressure). Once norepinephrine is administered to the monitored object, the monitored object's blood pressure reaches the target range within a time period, which is followed by a period of the continuous use of drug. It can be understood that the flow rate of the infusion pump 30 during this time period does not change freely in most cases. After a period of time, if the monitored object's blood pressure falls again and falls out of the target range, that is, systolic blood pressure is lower than 100 mmHg, or diastolic blood pressure is lower than 65 mmHg, the processor 12 controls to generate event information, for example, “the systolic blood pressure or the diastolic blood pressure is lower than the target range, pay attention to the amount of used norepinephrine”, and controls to display same in the alarm and prompt region 2. Otherwise if the monitored object's blood pressure is higher than the target range, that is, the systolic blood pressure is higher than 140 mmHg, or the diastolic blood pressure is higher than 105 mmHg, the processor 12 also controls to generate event information, for example, “the systolic blood pressure or the diastolic blood pressure is higher than the target range, pay attention to the amount of used norepinephrine”, and controls to display same in the alarm and prompt region 2. The processor 12 further provides prompt information that matches a prompt level of the above event information according to the prompt level, and at the same time, controls to highlight the norepinephrine in the display 13, to prompt the operator to consider a use strategy of the norepinephrine.

For another example, the operator uses an anti-arrhythmic drug for a monitored object with arrhythmia problems. The drug lidocaine is usually injected for the monitored object with arrhythmia such as ventricular premature beats or ventricular tachycardia. Under normal conditions, the monitored object's related arrhythmia problems disappear after the use of the lidocaine for a period of time. If such arrhythmia problems still exist, or such arrhythmia problems occur less frequently but still exist, it means that the drug is not effective for the monitored object, and other anti-arrhythmic drugs should be considered. The processor 12 controls to generate event information, for example, “ventricular tachycardia, pay attention to the use of lidocaine”, and controls the display 13 to display the event information in the alarm and prompt region 2 to inform the operator in time.

It can be understood that the above target range may also be replaced with a proportion by which a target value is exceeded, or an absolute value by which a target value is exceeded, for example, twenty percent of the target value.

Further, in one embodiment, the storage unit 15 further stores a predefined duration threshold for triggering generation of a prompt information by unusual fluctuation of each physiological sign parameter. The processor 12 controls to generate prompt information when duration of unusual fluctuation of any physiological sign parameter reaches a corresponding duration threshold. For example, a five-minute duration constraint is set for the event that the systolic blood pressure is higher than 140 mmHg or the diastolic blood pressure is higher than 105 mmHg, such that the processor 12 controls to generate event information only when the systolic blood pressure is higher than the threshold 140 mmHg for five minutes or the diastolic blood pressure is higher than 105 mmHg for five minutes. In this way, the interference caused by occasional transient fluctuation can be effectively avoided.

Further, in one embodiment, when it is determined, according to unusual fluctuation of a physiological sign parameter, that the current flow rate of the drug needs to be controlled, the processor 12 is further configured to generate a drug flow rate control instruction, and send the drug flow rate control instruction to the infusion pump 30 using the communication unit 14, so that the infusion pump 30 controls the flow rate of the drug of the infusion pump in response to the drug flow rate control instruction. Therefore, when it is determined, according to the unusual fluctuation of the physiological sign parameter, that the current flow rate of the drug needs to be controlled, the flow rate of the drug can be controlled in time, the workload of the operator can be reduced, and the intelligent management of patient monitoring and infusion can be achieved.

Further, in one embodiment, when it is determined, according to unusual fluctuation of a physiological sign parameter, that infusion needs to be started or stopped currently, the processor 12 is further configured to generate an infusion start or stop instruction, and control the communication unit 14 to transmit the infusion start or stop instruction to the infusion pump 30, so that the infusion pump 30 starts or stops the infusion in response to the instruction. Therefore, when it is determined, according to the unusual fluctuation of the physiological sign parameter, that the infusion needs to be started or stopped currently, a corresponding control operation of starting or stopping the infusion can be immediately performed in response to the instruction, thus avoiding accidents, and achieving the intelligent management of patient monitoring and infusion.

### Vital Sign and Medication Information Display Region 3

The processor 12 controls the display 13 to perform combined display of the physiological sign parameters and the medication information in the vital sign and medication information display region 3, so that the medical staff can view an association between a medication administration process and fluctuation of vital sign parameters, and help the medical staff pay attention to the effect of the use of drugs on the stability of the monitored object's vital sign parameters.

In one embodiment, the physiological sign parameters and the medication information are displayed in association in at least two manners. In a first manner: the two are displayed by time-based association, which means that a correlation between a physiological sign parameter trend and a medication flow rate trend or a dose/concentration trend is analyzed over time. For example, the processor 12 can be configured to control the display 13 to perform associated display of the physiological sign parameters and the medication information by: associating, by the processor 12, the physiological sign parameters with the medication information in time, and then controlling the display 13 to display the correlation between the physiological sign parameter trend and the medication flow rate trend or a dose/concentration trend over time. In a second manner, the physiological sign parameter and the medication information are displayed by distribution-based association, which means comparison of distribution of measured values of the physiological sign parameters in a period of time before and after medication. Specifically, the processor 12 can be configured to control the display 13 to perform associated display of the physiological sign parameters and the medication information by: associating, by the processor 12, the physiological sign parameters within a preset time period before medication with the physiological sign parameters within a preset time period after the medication, and then controlling the display 13 to display an association between the physiological sign parameters and the use of such the drug.

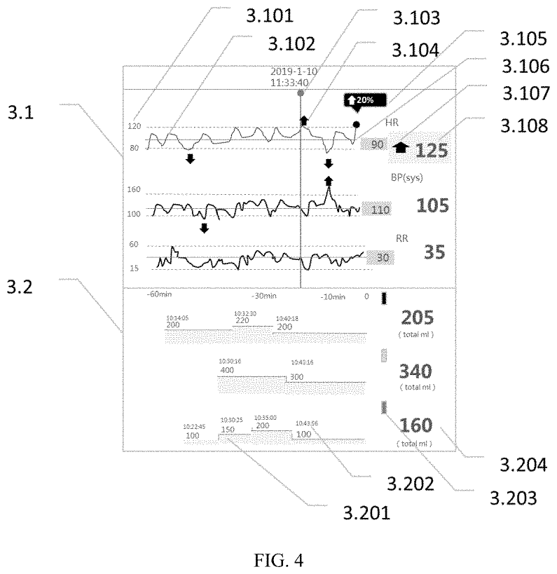

As shown in FIG. 4, FIG. 4 is a schematic diagram of combined display of physiological sign parameters and medication information that are associated based on time. A region 3.1 is used for a trend chart of measured values of the physiological sign parameters. A region 3.2 is used for a trend chart region of a drug flow rate (or dose/concentration). In some embodiment, the region 3.1 can be referred as a trend chart region, and the region 3.2 can be referred as a medication information region. That is, the vital sign and medication information display region may include a trend chart region for displaying the parameter information of the physiological sign parameter(s) and a medication information region for displaying the medication information, such as the information related to medication operation. In this figure, a region for a one-hour trend chart is displayed. It can be understood that a time interval length of the trend chart can be customized according to the needs of an operator.

In this example, in the region 3.1, a ruler line 3.101 can be maximum and minimum alarm limit thresholds, or a target range threshold; a trend curve 3.102 of a physiological sign parameter reflects a change of the physiological sign parameter over time; the operator can move a cursor 3.103 on a timeline to point to a certain time point; an element 3.104 refers to an event that a measured value exceeds the threshold in the past; an element 3.105 refers to an event that a measured value of the physiological sign parameter exceeds the threshold currently; an element 3.106 is a target value ruler which can be provided when the operator defines a specific target value instead of the target range Oct. 3, 2021, and then the processor 12 can calculate the target range threshold according to the defined threshold (such as a change of 20%); an arrow of an abnormality indicator icon 3.107 indicates the nature of an abnormality, that is, whether it is higher than the threshold or lower than the threshold, where the manner of indicating the abnormality is not limited to an icon, and the abnormality can also be indicated by color, or blinking color and measured value; an element 3.108 is a measured value at the time point selected by the cursor. In the region 3.1 of FIG. 4, one-hour trend curves of three physiological sign parameters, namely, a heart rate (HR), systolic blood pressure (BP-sys), and a respiratory rate (RR) are given. The operator can select several physiological sign parameters that need to be paid attention to according to his/her own needs.

In the region 3.2, an element 3.201 is a flow rate trend of the drug; an element 3.202 is a time at which the flow rate changes; and an element 3.203 is a drug name and a drug label color. Color-coded labels are usually used clinically to distinguish different types of drugs, for example, violet for vasopressor drugs and warm red for muscle relaxant drugs. An element 3.204 is a cumulative liquid volume of the drug in a time period. The flow rate trend may alternatively be replaced by a dose/concentration trend. In the region 3.2 of FIG. 4, flow rate trend curves of the three drugs dopamine, milrinone, and norepinephrine obtained by the monitor from the integrated infusion pump 30 are given. According to the number of integrated infusion pumps 30 and a specific drug name, the monitor can display flow rate (or dose/concentration) trends of more drugs.

The measured values of the physiological sign parameters and the drug flow rate trend are displayed on a timeline. Since a concentration of a prepared drug solution is fixed, a change of the flow rate means a change of a drug amount in a unit time, and a relationship between a segment of unusual fluctuation of the physiological sign parameter and the drug flow rate can be easily seen. When the cursor moves to a certain event position, a measured value of the physiological sign parameter and the drug flow rate information at that time point are displayed.

When the operator adjusts the flow rate of the medication administration, the effects of some drugs on the physiological sign parameters are very sensitive with a rapid response. For example, the effects of vasoactive drugs (for example, dopamine, milrinone, norepinephrine, etc.) on the blood pressure and the heart rate are remarkable and sensitive, and sedation and analgesia drugs (for example, lidocaine, as well as quinidine, metoprolol, amiodarone, verapamil, etc.) have remarkable and sensitive effects on consciousness, followed by the heart rate and the blood pressure. After adjusting the flow rate of the medication administration, the operator waits for a period of time beside the infusion pump 30 to observe a change trend of the monitored object's related physiological sign parameters and determine whether a current flow rate of the medication administration is appropriate. If the current flow rate of the medication administration is inappropriate, the current flow rate may be further adjusted, and if the current flow rate of the medication administration is appropriate, the current flow rate may be maintained.

Further, when at least one of the flow rate of the drug and the dose/concentration changes or a new drug is added, the processor 12 controls to generate a corresponding parameter curve or a mathematical expression of parameter value fluctuation, and controls the display 13 to display the parameter curve or the mathematical expression of parameter value fluctuation. The parameter curve is a curve formed by fluctuation of the physiological sign parameter over time. The mathematical expression of the parameter value fluctuation is a mathematical expression of the curve formed by the fluctuation of the physiological sign parameter, for example, a mathematical expression that forms the curve, or a key feature value that forms the curve, such as a slope.

Further, in one embodiment, when it is determined that the flow rate of the drug increases, the processor 12 controls the display 13 to display a watch window, where in the watch window, a change trend chart of physiological sign parameters closely related to the drug versus the flow rate of the drug within a preset time period is displayed, with a current flow rate value of the drug highlighted.

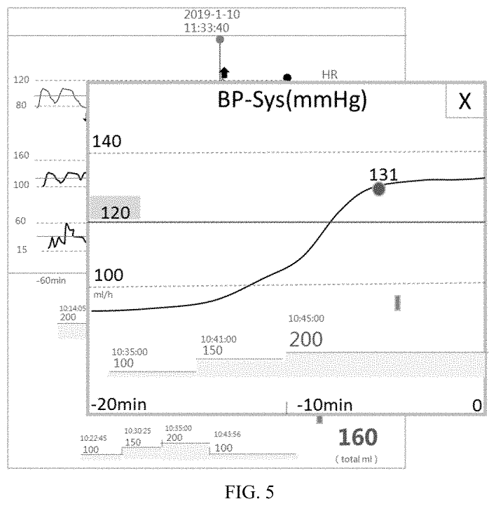

As shown in FIG. 5, in one implementation example, a change in a flow rate of norepinephrine is used to show a design that assists the operator in determining whether a flow rate of medication administration is appropriate. When determining that the flow rate of norepinephrine administration increases, the processor 12 controls the display 13 to display a watch window to display a trend chart of changes in the flow rate of the norepinephrine and the blood pressure within a preset event, with a current flow rate value highlighted. At this time, a systolic blood pressure (BP-Sys) trend is acquired with high-precision data, and an acquisition dot frequency is not less than one dot per second, so that changes in the blood pressure trend can be refreshed in time. When the flow rate is 100 mL/h, the monitored object's blood pressure is already below the target range, and then the operator increases the flow rate to 150 mL/h; the monitored object's blood pressure rises to a certain extent, but the rise is slow and not to the expectations, and then the operator further increases the flow rate to 200 mL/h; the operator empirically determines that a rise slope of a blood pressure curve is ideal at this time, and the blood pressure rise slows down shortly and begins to maintain in a relatively stable state. The systolic blood pressure value at a stable inflection point is 131 mmHg, reaching the blood pressure target, and thus the medication administration maintains at the flow rate of 200 mL/h subsequently.

Further, in one embodiment, the processor 12 is further configured to close the watch window 11 in response to a close operation of a user, or the processor 12 controls to close the watch window 11 when the flow rate of such a drug no longer changes within the preset time period.

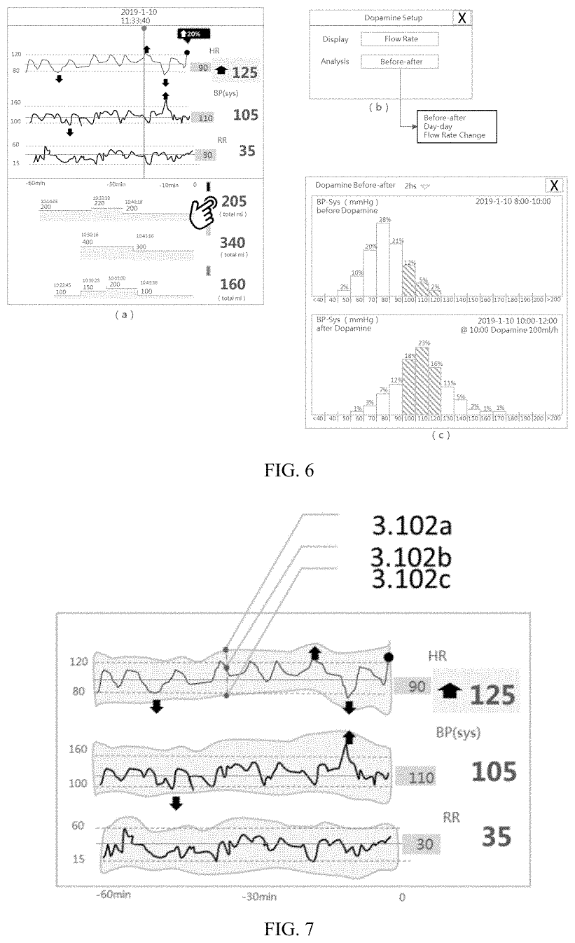

Further, in one embodiment, as shown in FIG. 6, FIG. 6 is a schematic diagram of combined display of physiological sign parameters and medication information that are associated based on distribution. In response to an operation of clicking a dopamine area in a window (a), the processor 12 controls to open a “Dopamine Setup” menu (b), and controls to select whether to display the flow rate trend or the dose/concentration trend in the menu, and select an analysis method. The analysis method includes, but is not limited to, before-after medication comparison, day-day comparison, and flow rate change. A window (c) is an analysis result of the before-after medication of dopamine. In the window (c), dopamine was administered to the monitored object at Jan. 10, 2019 10:00. An upper part of the window (c) is a statistical distribution diagram of the measured values of the systolic blood pressure 2 hours before the use of the drug dopamine, and a lower part is a statistical distribution diagram of the measured values of the systolic blood pressure 2 hours after the use of the drug dopamine. The statistical duration can be modified by the operator. It can be seen from the figure that with the use of the drug dopamine, the proportion of the original hypotensive monitored object's blood pressure in the target range of the blood pressure began to increase. It can also be understood that the blood pressure of the monitored object is mainly distributed in 61 mmHg to 90 mmHg (accounting for 69%, in other words, 69% of the blood pressure falling within such a blood pressure range in terms of time) 2 hours before the medication, while only 19% of the blood pressure falls within the target range. With the use of the drug, the proportion of the monitored object's blood pressure falling within the target range within 2 hours after the medication increased to 57%.

Further, in one embodiment, the processor 12 can be configured to control the display 13 to perform associated display of the physiological sign parameters and the medication information by: presenting the physiological sign parameters by a max-min-average trend chart, where the max-min-average trend chart is formed by: obtaining maximum values, minimum values, and average values in a plurality of values of the physiological sign parameters measured in each unit time within the preset time period, and then connecting all the maximum values, all the minimum values, and all the average values within the preset time period to form respective curves, so as to form the max-min-average trend chart representing the physiological sign parameter trend, where the preset time period includes multiple unit times.

In addition to the average value trend chart (element 3.102) shown in FIG. 4, which is a line obtained by connecting the average values in the unit times, a trend chart of the measured values of the physiological sign parameters in the region 3.1 may be a maximum-minimum value trend chart. With the development of high-speed data collection, the physiological data monitoring apparatus 11 can obtain a plurality of data values within several extremely small time periods. However, limited by the actual number of pixels of the display 13 in a limited area, it may not be viable to show all points truthfully. In this case, one pixel may represent one unit of time (such as 1 second, 1 minute, and 5 minutes) to obtain three of the values measured in the units of time, namely, the maximum value, the minimum value, and the average value. Then, all the maximum values, all the minimum values, and all the average values within the preset time period are connected to for respective curves, to form the max-min-average trend chart.

FIG. 7 is a max-min-average trend chart according to an embodiment of the disclosure. Elements 3.102a, 3.102b, and 3.102c are three values in the same time unit. The element 3.102a is a maximum value in the unit time, the element 3.102b is an average value in the unit time, and 3.102c is a minimum value in the unit time. In this way, the operator can clearly see a fluctuation range of the monitored object's vital sign values, which has good guiding significance for the use of an area to display a trend curve for a long time, and avoids a case where a fluctuation status of the monitored objects with frequently fluctuated vital sign values is ignored in the case of only an average value.

However, no matter it is the average value trend curve or the maximum-minimum value trend curve mentioned above, an interference removal method can also be used to remove extremely abnormal measured values before averaging or selection of the maximum and minimum values is performed. Clinically extremely abnormal measured values do not reflect a real condition of the monitored object's vital signs, and instead, interference introduced by some factors leads to abnormally large deviation from the measured values.

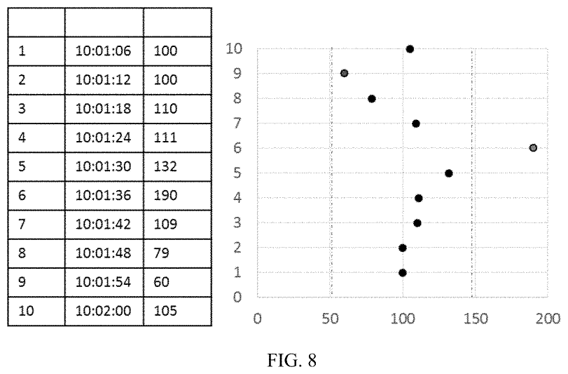

FIG. 8 is a principle diagram of removing an abnormal value according to an embodiment of the disclosure. Taking 10 pieces of heart rate data collected by the monitor within 1 minute as an example, the data numbered 6 in the 10 pieces of data is 190, which is abnormally high. Therefore, the 6^{th} piece of data is removed, only 9 pieces of data between the two dashed lines are used to calculate an average value, the data numbered 5 is selected as a maximum value (132), and the data numbered 8 is selected as a minimum value (79). The dashed lines can be selected according to fluctuation rules of the physiological sign parameters of a human body, and it may be considered that a proportion of abnormal values shall not exceed the percentage of a data amount in a unit time, such as 90%.

Further, in one embodiment, the monitor collects statistics on infusion amounts of different drugs infused into the body of the monitored object in each time period to obtain the cumulative infusion amounts of different drugs in different time periods. Therefore, an overall trend of an infused volume for the monitored object, such as a liquid infusion trend, within 1 hour, 8 hours, and 24 hours, can be observed.

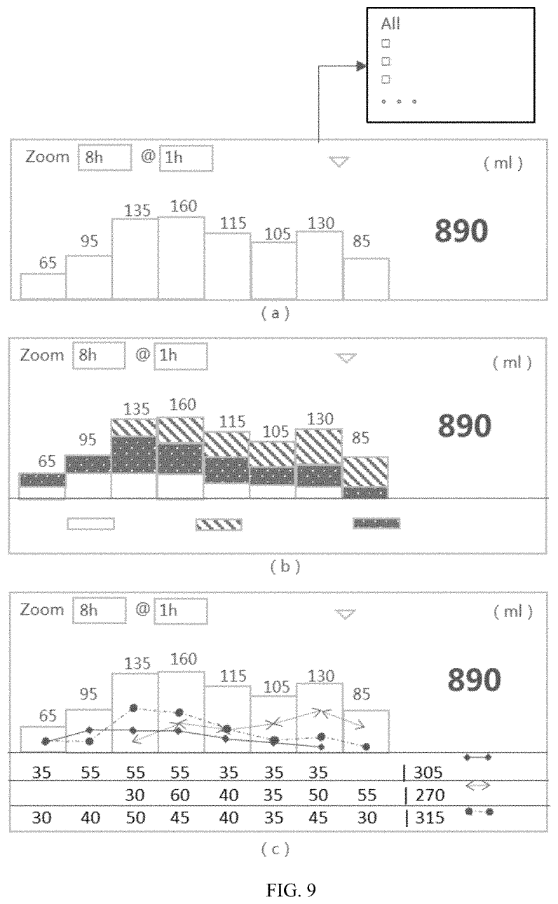

FIG. 9 is an example diagram of a liquid infusion statistical result, showing the liquid infusion within 8 hours. The operator can select a time period and a sub-time period represented by each histogram. A time period shown in FIG. 9(a) is 8 hours, and each histogram represents 1 hour. The operator can also manually select drugs that need to be performed with the statistical collection, for example, all or several drugs, such as statistical collection on an adrenaline dose/liquid infusion amount, on a dopamine dose/liquid infusion amount, or on adrenaline, norepinephrine, and dopamine dose/liquid infusion amounts shown in FIGS. 9(b) and 9(c). In FIG. 9(b), trend display for different drugs is added, and in FIG. 9(c), a cumulative trend of different drugs and a cumulative infusion amount in a unit time are added.

### Event Overview Region 5

Further, the processor 12 collect statistics on types of abnormality events, the number of occurrences of abnormality events, and key indicators in abnormality events within a preset time period, and controls the display 13 to display, in the event overview region 5, the types of abnormality events, the number of occurrences of abnormality events, and the key indicators in the abnormality events within the preset time period. For example, statistics on the total number of occurrences of arrhythmia events within 8 hours, and the number of occurrences of different arrhythmia events may be collected, such as “ventricular fibrillation (1)” and “excessively high HR (3)”. At the same time, detailed information, such as “ventricular fibrillation @ 10:54 for 55 seconds”, “excessively high HR @ 10:03, @ 10:18, and @ 10:41, Max 135, and Min 87” can be viewed. With the information, the operator can view details of the event in the vital sign and medication information display region 3, as well as a trend of the parameters for a time period before and after the event.

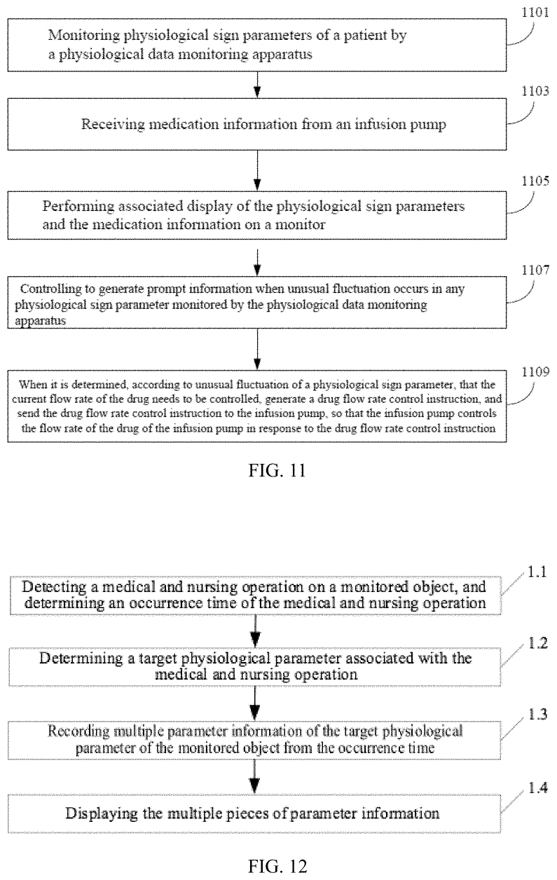

Referring to FIG. 11, FIG. 11 is a flowchart of a method, applied to a monitor, for combined display of physiological sign parameters and medication information according to an embodiment of the disclosure. The execution order of the method is not limited to the order shown in FIG. 11. The method for the combined display of the physiological sign parameters and the medication information includes the steps as follows.

At step 1101: physiological sign parameters of a monitored object are monitored. Specifically, the physiological data monitoring apparatus 11 of the monitor 10 monitors the physiological sign parameters of the monitored object.

At step 1103: medication information from the infusion pump 30 is received. Specifically, the communication unit 14 of the monitor 10 receives the medication information from the infusion pump 30.

At step 1105: associated display of the physiological sign parameters and the medication information on the monitor 10 is performed. The processor 12 of the monitor 10 controls to perform associated display of the physiological sign parameters and the medication information on the display 13.

The medication information includes at least one of a flow rate of a drug, a dose, a concentration, a cumulative infused liquid volume, and a physiological sign parameter that is mainly affected during the use of the drug, and the physiological sign parameter that is mainly affected during the use of the drug is at least one of the monitored physiological sign parameters.

Further, in one embodiment, the method further includes the following step. At step 1107: it is controlled to generate prompt information when unusual fluctuation occurs in any physiological sign parameter monitored by the physiological data monitoring apparatus 11.

Further, in one embodiment, the step 1107 includes: determining and controlling to generate prompt information corresponding to a prompt level according to a predefined correspondence between a fluctuation range of each physiological sign parameter and a prompt level and a current range of unusual fluctuation of the physiological sign parameter.

Further, in one embodiment, the step 1107 includes controlling to generate the prompt information when duration of unusual fluctuation of any physiological sign parameter reaches a corresponding duration threshold, where the duration threshold is a predefined duration threshold for triggering generation of a prompt information by unusual fluctuation of each physiological sign parameter.

Further, in one embodiment, the method further includes the following step:

At step 1109: when it is determined, according to unusual fluctuation of a physiological sign parameter, that the current flow rate of the drug needs to be controlled, a drug flow rate control instruction is generated, and the drug flow rate control instruction is sent to the infusion pump 30, so that the infusion pump 30 controls the flow rate of the drug of the infusion pump in response to the drug flow rate control instruction.

Further, in another embodiment, the step 1109 may include generating an infusion start or stop instruction when it is determined, according to unusual fluctuation of a physiological sign parameter, that infusion needs to be started or stopped currently, and controlling to send the infusion start or stop instruction to the infusion pump 30, so that the infusion pump 30 starts or stops the infusion in response to the instruction.

Further, in one embodiment, the method further includes a step of:
when at least one of the flow rate of the drug and the dose/concentration changes or a new drug is added, controlling to generate and display a corresponding parameter curve or a mathematical expression of parameter value fluctuation.

Further, in one embodiment, the method further includes a step of:
when it is determined that the flow rate of the drug increases, controlling to display a watch window, where in the watch window, a change trend chart of a physiological sign parameter closely related to the drug versus the flow rate of the drug within a preset time period is displayed, with a current flow rate value of the drug highlighted.

Further, in one embodiment, the method further includes a step of:
closing the watch window in response to a close operation of a user; or
controlling to close the watch window when the flow rate of the drug no longer changes within the preset time period.

Further, in one embodiment, the associated display of the physiological sign parameters and the medication information on the monitor includes:
associating the physiological sign parameters with the medication information in time, and then displaying on the monitor a correlation between the physiological sign parameter trend and a medication flow rate trend or a dose/concentration trend over time.

Further, in one embodiment, the associated display of the physiological sign parameters and the medication information on the monitor includes:
associating the physiological sign parameters within a preset time period before medication with the physiological sign parameters within a preset time period after the medication, and then displaying on the monitor an association between the physiological sign parameters and the use of the drug.

Further, in one embodiment, the method further includes a step of:
presenting the physiological sign parameters by a max-min-average trend chart.

Further, in one embodiment, the max-min-average trend chart is formed by:
obtaining maximum values, minimum values, and average values in a plurality of values of the physiological sign parameters measured in each unit time within the preset time period; and
connecting all the maximum values, all the minimum values, and all the average values within the preset time period to form respective curves, so as to form the max-min-average trend chart representing a physiological sign parameter trend, where the preset time period includes a number of unit times.

Further, in one embodiment, the method further includes a step of:
collecting statistics on infusion amounts of different drugs infused into the body of the monitored object in each time period to obtain cumulative infusion amounts of different drugs in different time periods.

Further, in one embodiment, the method further includes a step of:
collecting statistics on types of abnormality events, the number of occurrences of the abnormality events, and key indicators in the abnormality events within a preset time period, and controlling to display the statistics.

Therefore, the monitor can perform associated display of the physiological sign parameters of the monitored object and the medication information, such that an association between the physiological sign parameters of the monitored object and the medication information can be presented in a more comprehensible form, to help physicians analyze an association between a medication administration process and fluctuation of a vital sign value, and also make it convenient for nurses to perform bedside medication administration.

In the medical field, after performing some medical and nursing operations for a monitored object, a medical staff pays more attention to the implementation effect of the medical and nursing operations, and therefore needs to monitor changes in vital signs of the monitored object. In some cases, a medical staff performs bedside observation with a medical device, the medical device displays real-time monitoring values of physiological sign parameters of a monitored object, and the medical staff determines the effects of the medical and nursing operations by means of observing changes in the real-time monitoring values. Such a monitoring method has a waste of labor costs, and requires the medical staff to perform bedside observation personally at all times, resulting in poor experience.

The disclosure provides a method for displaying physiological sign parameter, which can record and display multiple physiological sign parameters after medical and nursing operations to provide visual vital sign change information for the medical staff, which reduces the cost of personal clinical observation.

The physiological sign parameter display method can be applied to various medical devices, including but not limited to, a ventilator, an anaesthesia machine, a defibrillator, an electrocardiograph, a telemetering device, a central station, and a vital sign monitoring device such as a monitor. Referring to FIG. 12, the method can specifically include steps 1.1-1.4.

At step 1.1, a medical and nursing operation on a monitored object is detected, and an occurrence time of the medical and nursing operation is determined.

After the medical staff performs the medical and nursing operation on the monitored object, the medical device can detect the occurrence of this event. After detecting the medical and nursing operation performed on the monitored object, the medical device first needs to determine the occurrence time of the medical and nursing operation.

The medical and nursing operations may include medical operations and/or nursing operations. In some examples, the medical and nursing operations may include various operations of, for example, nursing operations such as intubation and back slapping performed for the monitored objects, for another example, therapeutic operations such as mechanical ventilation performed for the monitored objects, for still another example, medication related operations performed for the monitored objects, etc. The medication related operations may include: administering the monitored object with a drug, and adjusting medication situations of the used drug of the monitored object, e.g., adjusting the flow rate, adjusting the drug volume, adjusting the concentration, etc.

In an example, the medical device can determine, by the following methods, that the medical and nursing operation has been performed on the monitored object. In one of the methods, after performing the medical and nursing operation, a user inputs related information of the medical and nursing operation into the medical device. In another method, the medical staff needs to perform the medical and nursing operation on the to monitored object by means of another device (which can be referred to as an execution device), and the medical device is connected to the execution devices so as to determine whether there is a medical and nursing operation on the monitored object by means of monitoring whether the execution device has an event associated with the medical and nursing operation.

For example, when an infusion pump is used to inject a drug into the monitored object, the medical staff can adjust a flow rate, a drug volume, a concentration, etc. of the used drug; when detecting these operation events, the infusion pump can send the related information of the operation events to the medical device; and after receiving the related information of the operation events, the medical device can determine that the monitored object is subjected to the medical and nursing operation.

In addition, the methods by which the medical device determines the occurrence time of the medical and nursing operation may also include the following two methods.

In one of the methods, the medical device can receive the occurrence time that is input by the user for the medical and nursing operation. Specifically, after the medical device detects the medical and nursing operation, the medical staff can input the occurrence time point of the medical and nursing operation to the medical device by means of an input interface or input keys.

In the other method, the execution device for the medical and nursing operation is determined, and the occurrence time of the medical and nursing operation is acquired from the execution device. Specifically, the execution device that performs the medical and nursing operation can automatically record the occurrence time of the medical and nursing operation and send the occurrence time to the medical device connected to the execution device.

At step 1.2, a target physiological sign parameter associated with the medical and nursing operation is determined.

The physiological sign parameter associated with the medical and nursing operation is also referred to as the target physiological sign parameter.

The physiological sign parameter associated with the medical and nursing operation refers to the physiological sign parameter that is affected by the medical and nursing operation or refers to the physiological sign parameter that can reflect the effect of the medical and nursing operation. For example, the medical and nursing operation is administering the monitored object with the drug “dopamine”. The drug “dopamine” may affect monitoring values of the physiological sign parameters, such as the heart rate, the respiration rate, the blood pressure, etc., of the monitored object, and therefore those physiological sign parameters are taken as the target physiological sign parameters.

Specifically, the methods for determining the target physiological sign parameters may include the following methods.

In one example, a physiological sign parameter type that is input by the user for the medical and nursing operation is received and taken as the target physiological sign parameter; or the target physiological sign parameter corresponding to the detected medical and nursing operation is determined according to a preset correspondence between the medical and nursing operation and the physiological sign parameter; or parameter information of an optional physiological sign parameter is obtained within a preset time period, and the optional physiological sign parameter, which has the change trend of the parameter information meeting a preset condition, is taken as the target physiological sign parameter.

Regarding the second method, it may be noted that the preset correspondence may be set on the medical device, and after detecting the medical and nursing operation, the medical device searches the physiological sign parameter corresponding to the medical and nursing operation according to the preset correspondence, and the searched physiological sign parameter is used as the target physiological sign parameter. The physiological sign parameters corresponding to different medical and nursing operations can be the same or different.

Regarding the third method, it may be noted that some optional physiological sign parameters can be preset. After the medical and nursing operation is detected, parameter information of the optional physiological sign parameters within a preset time period length is recorded, the change trend of the parameter information of each optional physiological sign parameters is obtained according to the recorded content, and it is determined whether the change trend of the parameter information meets the preset condition. When the change trend of certain parameter information meets the preset condition, the optional physiological sign parameter corresponding to the parameter information is determined as the target physiological sign parameter. The preset condition can represent the effect of the medical and nursing operation, for example, the preset condition is that the amplitude of change in the parameter information exceeds a certain range. to At step 1.3, multiple parameter information of the target physiological sign parameter of the monitored object is recorded from the occurrence time.

The medical device starts to record the parameter information of the target physiological sign parameter after determining the occurrence time of the medical and nursing operation and determining the target physiological sign parameter that needs to be monitored.

The medical device records the parameter information of the target physiological sign parameter from the occurrence time. The parameter information can be specifically a parameter value, and can also be other information that can reflect the target physiological sign parameter. It may be noted that there are multiple parameter information, and in the case that there are multiple target physiological sign parameters, each of the target physiological sign parameters also corresponds to multiple parameter information.

The medical device can record continuously, that is, the medical device can keep recording the parameter information of the target physiological sign parameter of the monitored object from the occurrence time until a recording stop instruction is received. For example, after detecting the medical and nursing operation, the medical device records the parameter information, from the occurrence time of the medical and nursing operation, until the user inputs the recording stop instruction to the medical device. As can be seen, in this recording method, the medical staff determines the time length of collecting the parameter information, and it may be noted that in the case that there are multiple target physiological sign parameters, the recording stop instruction can be directed to one or more of the multiple target physiological sign parameters.

In the other recording method, the recording can be automatically stopped according to the preset time length. That is, the medical device keeps recording the parameter information of the target physiological sign parameter of the monitored object, from the occurrence time within the preset time period. Specifically, the medical device presets the time period lengths corresponding to the target physiological sign parameters, and the time period lengths that the same medical and nursing operation takes the action for different target physiological sign parameters may be different;

therefore, the time period lengths corresponding to the different target physiological sign parameters may be different. The value of the preset time period may be any value set according to medical experience, which is not specifically defined in the disclosure.

At step 1.4, the multiple parameter information is displayed.

In an interface displayed by the medical device, the multiple parameter information recorded is displayed. It may be noted that the interface that displays the parameter information can only contain the multiple parameter information, or can also contain other parameter information besides the multiple parameter information. For example, the other parameter information may include real-time values of the target physiological sign parameter or real-time values of other physiological sign parameters, real-time waveforms of the target physiological sign parameter, etc.

An exemplary display method in this step may include listing the multiple parameter information in the display interface according to the sequence of recording time, so that the user can view the change in the parameter information with the time.

Another exemplary display method in this step may include: generating a trend chart and/or an waveform chart corresponding to the medical and nursing operation according to the multiple parameter information; and displaying the trend chart and/or the waveform chart corresponding to the medical and nursing operation. Specifically, the parameter information may include parameter values. In a two-dimensional coordinate system formed by a time coordinate axis and a monitoring value coordinate axis, the parameter values of the target physiological sign parameter are arranged according to the sequence of collection time points so as to form the trend chart and/or the waveform chart of the target physiological sign parameter.

It may be noted that in the case that there are multiple target physiological sign parameters, the graph formed by some target physiological sign parameter (s) is referred to as the trend chart, and a graph formed by some target physiological sign parameter(s) is referred to as the waveform chart. For ease of differentiation from other trend charts and/or waveform charts, the trend chart and/or the waveform chart generated in the disclosure can be referred to as the trend chart and/or the waveform chart corresponding to the medical and nursing operation. For example, if the medical and nursing operation is a medication operation, the trend chart and/or the waveform chart can be referred to as a medication trend chart and/or waveform chart.

As can be seen, in such display methods, multiple parameter information recorded are displayed by a graph. The graph display method is more visual and is convenient for the user to view.

It may be noted that, before the medical device displays the trend chart and/or the waveform chart corresponding to the medical and nursing operation, other trend charts and/or waveform charts may be displayed in the display interface. For ease of differentiation by the user, the trend charts and/or waveform charts currently displayed can be switched to the trend chart and/or the waveform chart corresponding to the medical and nursing operation.

In an implementation, the review trend chart and/or review waveform chart currently displayed can be switched to the trend chart and/or the waveform chart corresponding to the medical and nursing operation.

The medical device currently displays the review trend chart and/or the review waveform chart, and the review trend chart and/or the review waveform chart records information of the physiological sign parameter of the monitored object within a historical time period. The length of the historical time period can be set by the user, for example, can be the information of the physiological sign parameter within one hour in history. In addition, the historical time period can be a previous historical time period calculated from the current time point, and may also be a previous historical time period calculated from a certain historical time point. The review trend chart and/or the review waveform chart can be refreshed in real time.

If the medical device has detected the occurrence of the medical and nursing operation, the medical device records the parameter information of the target physiological sign parameter associated with the medical and nursing operation in real time, generates a trend chart and/or a waveform chart from the parameter information, and switches the currently displayed review trend chart and/or review waveform chart to the generated trend chart and/or waveform chart.

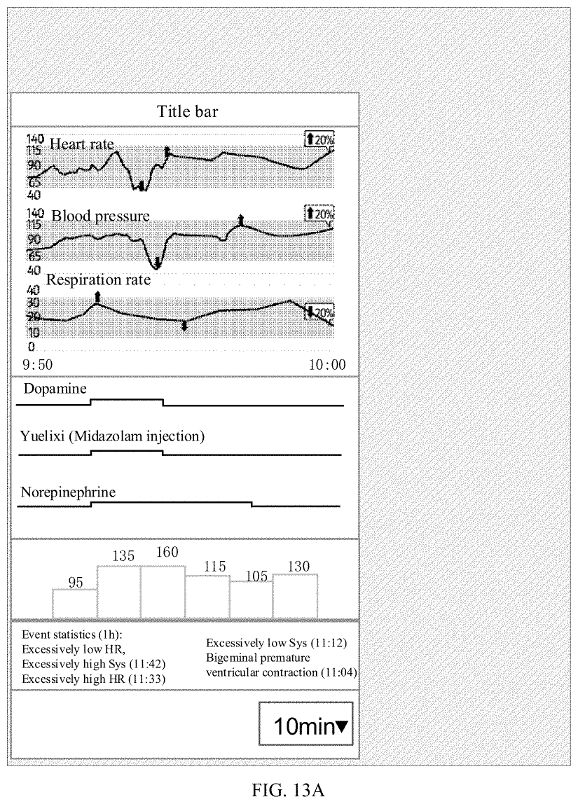

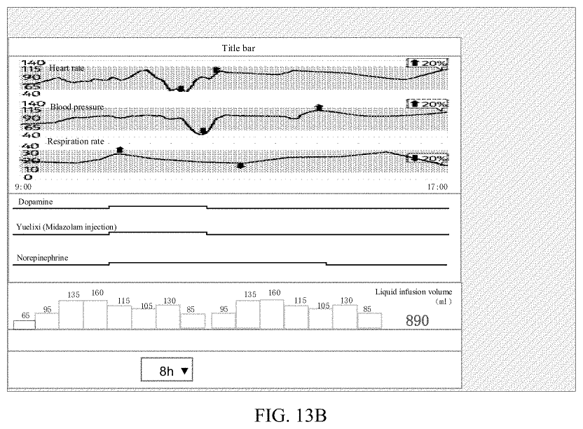

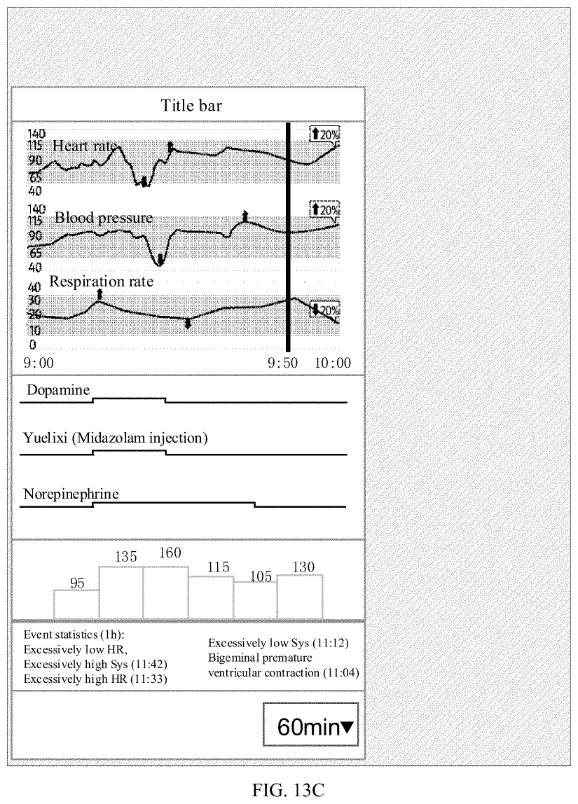

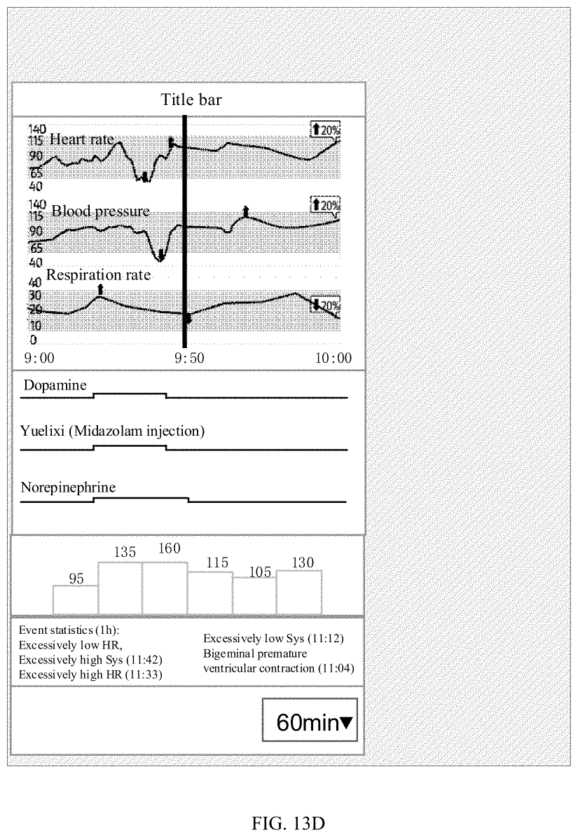

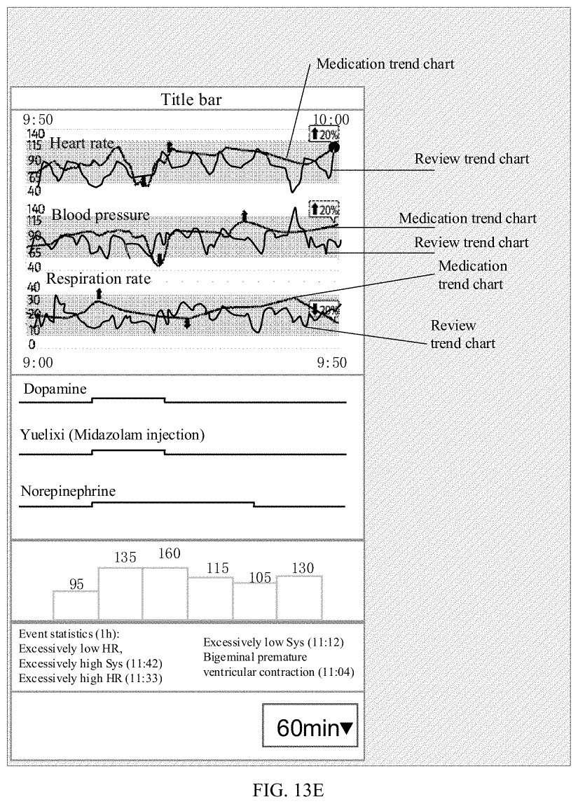

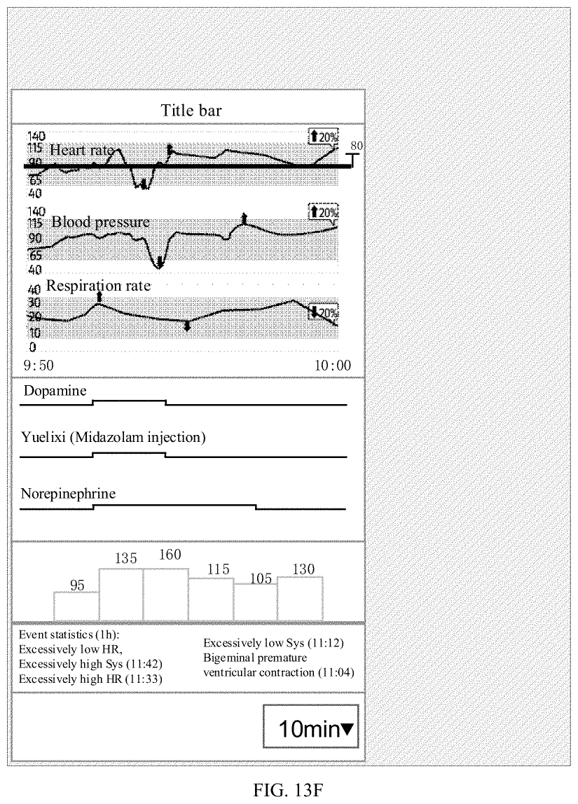

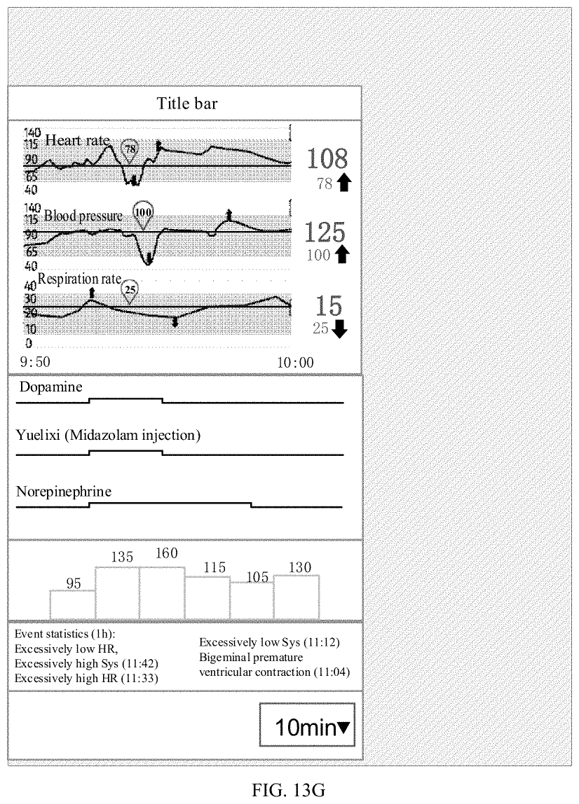

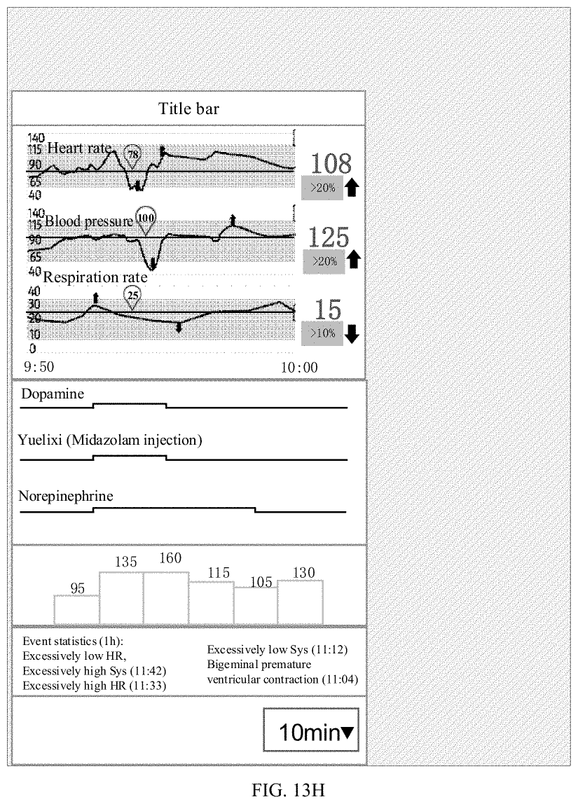

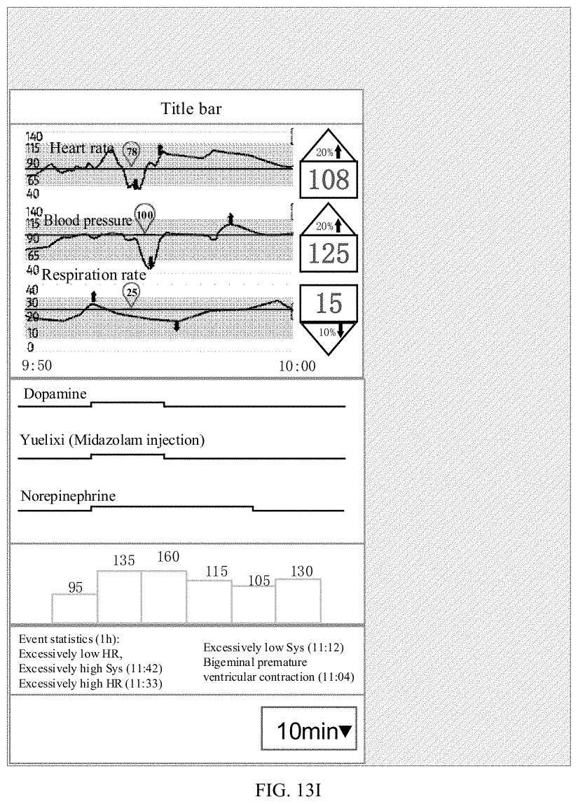

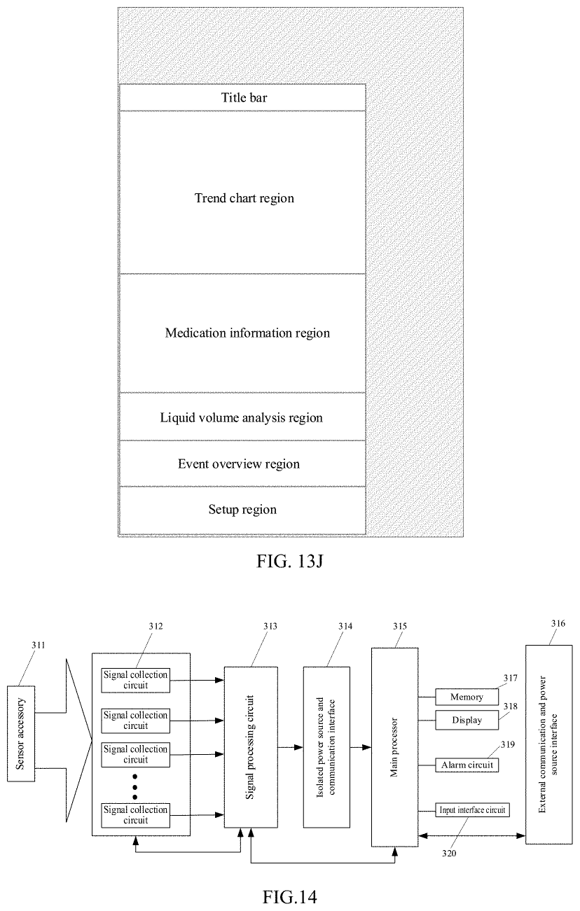

For example, assuming that the medical device keeps displaying a review trend chart of some physiological sign parameters of a monitored object who is administered, at a certain time point, with the drugs “dopamine”, “Yuelixi (midazolam injection)” and “noradrenaline” which may affect three physiological sign parameters, including the heart rate, the respiration rate and the blood pressure, of the monitored object as can be understood, the medical device switches the current review trend chart to the trend chart corresponding to the medication operation. As shown in FIG. 13A, the trend chart corresponding to the medication operation specifically comprises a heart rate (HR) trend chart, a blood pressure (BP) trend chart, and a respiration rate (RR) trend chart, which respectively show the change trends of the heart rate, the blood pressure and the respiration rate within a time period, such as 10 minutes, from the medication time point. It should be noted that the shadow portions in FIG. 13A indicate other monitoring information that can be contained in the display interface, such as real-time values of the above three physiological sign parameters or other physiological sign parameters described above.

It may be noted that, in addition to switching the review trend chart and/or the review waveform chart to the trend chart and/or the waveform chart corresponding to the medical and nursing operation, the trend chart and/or the waveform chart corresponding to the medical and nursing operation can also be switched to the review trend chart and/or the review waveform chart, and the switching condition may be receiving a switching instruction or display time length reaches a preset time length.

Moreover, the display time length of the trend chart and/or the waveform chart corresponding to the medical and nursing operation can be set by the user. As shown in FIG. 13A, the bottom setup region contains a time length setup button, and the time length set in the graph is 10 min (minutes), so the time length of the displayed trend charts of the three physiological sign parameters is 10 minutes from 9:50 to 10:00. Alternatively, the time length of the trend charts of the physiological sign parameters can be set in the setup button. For example, the time length is set to 8 h (hours), and the displayed trend charts of the physiological sign parameters are as shown in FIG. 13B, with the start point of the time length being 9:00, and the end point of the time length being 17:00 (the current time point).

As can be seen, in the above display method, in the display interface, the review trend chart and/or the waveform chart is replaced with the trend chart and/or the waveform chart of the medical and nursing operation, which not only reduces the occupied space of the display interface, but also enables the medical staff to easily obtain the relevant situation of the medical and nursing operation to prevent the interference of other information.

In another implementation, the trend chart and/or the waveform chart corresponding to the medical and nursing operation can be displayed concurrently together with the review trend chart and/or the review waveform chart. That is to say, the display interface of the medical device includes both the review trend chart and/or the review waveform chart and the trend chart and/or the waveform chart corresponding to the medical and nursing operation, both of which can be either displayed in the same region of the display interface or displayed in different regions of the display interface. The solution for simultaneous display may specifically include the following methods.

In a first method, the trend chart and/or the waveform chart corresponding to the medical and nursing operation are/is marked on the whole displayed review trend chart and/or review waveform chart. Specifically, the whole review trend chart and/or review waveform chart includes the trend chart and/or the waveform chart corresponding to the medical and nursing operation, and in order to remind the user, the trend chart and/or the waveform chart corresponding to the medical and nursing operation can be marked for prompting. A marking method may include adding, in the review trend chart and/or the review waveform chart, a time mark line that indicates the occurrence time point of the medical and nursing operation, so as to indicate that the review trend chart and/or the review waveform chart starts from the time mark line, and the trend chart and/or the waveform chart after the time point is the trend chart and/or the waveform chart corresponding to the medical and nursing operation. Another method may include setting a display pattern of the trend chart and/or the waveform chart corresponding to the medical and nursing operation to be different from that of the review trend chart and/or the review waveform chart. For example, the display pattern may include line color, line thickness, background color, etc.

In a schematic diagram of a display interface as shown in FIG. 13C, a heart rate trend chart, a blood pressure trend chart and a respiration rate trend chart are contained in a trend chart region. The three trend charts can be considered as review trend charts, with the time length being from 9:00 to 10:00. Assuming that the last monitored medication operation occurred at 9:50, a time mark line in the form of a vertical line is added at the position corresponding to the time point to prompt the medical staff of the occurrence time point of the last medication operation, such that the medical staff can understand that the trend chart after the vertical line is the trend chart corresponding to the medication operation.

In a second method, the trend chart region in the physiological sign parameter interface is divided into a first sub-region and a second sub-region according to a preset region proportion. The review trend chart and/or the review waveform chart is displayed in the first sub-region. The trend chart and/or the waveform chart corresponding to the medical and nursing operation is displayed in the second sub-region. Specifically, the physiological sign parameter interface of the medical device contains a trend chart region, which is a region for displaying the trend chart of the physiological sign parameter. Since the review trend chart and/or the review waveform chart needs to be displayed concurrently with the trend chart and/or the waveform chart corresponding to the medical and nursing operation, it is possible to preset the region proportion which is used to indicate the areas of the respective regions occupied by two types of trend charts and/or waveform charts. The trend chart region can be divided into two sub-regions according to the preset region proportion, and then the two types of trend charts and/or waveform charts can be respectively displayed in the two sub-regions. It can be noted that the specific value of the preset region proportion is not specifically defined in the disclosure.

It should be noted that the two trend charts may be different in time length. As shown in FIG. 13D, assuming that the preset region proportion is 1:1, the left half portion of the trend chart region shows the review trend chart, and the right half portion shows the trend chart corresponding to the medication operation. The occurrence time point of the last medication operation is 9:50, and assuming that the current time point is 10:00, the time length of the trend chart corresponding to the medication operation is 10 minutes. The time length of the review trend chart is 50 minutes from 9:00 to 9:50. Of course, the preset region proportion may also be other values such as 2:1.

In a third method, in the trend chart region of the physiological sign parameter interface, two vertically overlapped layers are used to respectively display the review trend chart and/or the review waveform chart and the trend chart and/or the waveform chart corresponding to the medical and nursing operation. Specifically, in the trend chart region of the physiological sign parameter interface, two types of trend charts and/or waveform charts are displayed in an overlapped manner. It should be noted that the two types of trend charts and/or waveform charts may have different time lengths. In addition, in order to distinguish the two types of trend charts and/or waveform charts, different display patterns may be set, for example, different colors, brightness, etc. are set.

As shown in FIG. 13E, each type of physiological sign parameter trend chart in the trend chart region contains a medication trend chart (that is, the trend chart corresponding to the medication operation) and a review trend chart. In this region, the upper time length (9:50-10:00) is the time length corresponding to the medication trend chart, and the lower time length (9:00-9:50) is the time length corresponding to the medication trend chart.

In a fourth method, a floating interface is popped up at an associated position, such as the right side, of the trend chart region in the physiological sign parameter interface, where the trend chart region is configured to display the review trend chart and/or the review waveform chart, and the trend chart and/or the waveform chart corresponding to the medical and nursing operation is displayed in the floating interface. Specifically, the pop-up operation of the floating interface can be based on a triggering operation of the user. For example, the user can click any position in the review trend chart to trigger displaying of the floating interface. The display position of the floating interface can be changed based on a drag operation by the user, and the floating display can also be cancelled based on a closing operation by the user.

As can be seen, the disclosure provides the above four solutions for displaying the review trend chart and/or the review waveform chart concurrently with the trend chart and/or the waveform chart corresponding to the medical and nursing operation. Of course, other methods that could be expected by those skilled persons in the art belong to the scope of protection of the disclosure.

It can be seen from the above solutions that the physiological sign parameter display method provided in the disclosure can detect the medical and nursing operation on the monitored object, determine the occurrence time of the medical and nursing operation, determine the target physiological sign parameter associated with the medical and nursing operation, record multiple parameter information of the target physiological sign parameter of the monitored object from the occurrence time, and display the multiple parameter information. In this way, the user can visually view changes in parameter information of the physiological sign parameter of the monitored object after performing the medical and nursing operation without observing real-time values of the physiological sign parameter beside the monitored object, resulting in good user experience.

In practical applications, after performing the medical and nursing operation, the medical staff not only needs to view the affection of the medical and nursing operation on the physiological sign parameters, but also pays attention to whether the amplitude of change of the affected physiological sign parameters reaches a certain degree. Therefore, alarm monitoring can be performed on the parameter information of the physiological sign parameter.

Therefore, on the basis of displaying the physiological sign parameter information, the physiological sign parameter display method may further include: determining an alarm condition corresponding to the target physiological sign parameter; determining, in the multiple parameter information displayed, the parameter information that meets the alarm condition, and prompting the parameter information that meets the alarm condition.

In an embodiment, the alarm condition corresponding to the target physiological sign parameter indicates whether the amplitude of change of the target physiological sign parameter reaches the level of attention of the user. The parameter information can be a parameter value. The alarm condition in one way is to determine whether the amplitude of change of the parameter value before and after the medical and nursing operation exceeds a certain threshold value. Alternatively, the alarm condition in another way is that the target physiological sign parameter is provided with a preset target parameter value, where the target parameter value is used to indicate a target situation that the parameter value of the target physiological sign parameter is expected to reach after the medical and nursing operation is performed on the monitored object, and the alarm condition is that a proportion of a difference value between the parameter value of the target physiological sign parameter and the target parameter value to the target parameter value is higher than or lower than a preset proportion value.

For example, the monitored object is administered with a certain drug, the medical staff expects that the heart rate value of the monitored object could reach 78 bpm (the target parameter value) after medication of the drug, and at the same time the medical staff also pays attention to whether the difference value between the heart rate parameter value and 78 after medication of the drug is above or below 20% of 78 (the preset proportion value). Therefore, if the difference value between the heart rate parameter value of the monitored object and 78 is above or below 20% of 78, it is necessary to have a prompt.

It should be noted that different target physiological sign parameters can correspond to different target parameter values and different preset proportion values. For example, the target parameter value corresponding to the heart rate is 78, and the corresponding preset proportion value is 20%; and the target parameter value corresponding to the respiration rate is 25, and the corresponding preset proportion value is 10%. Of course, the numerical values here are merely exemplary and may also be set to other values as desired in practical applications.

Further, the parameter values that meet the alarm condition can also be marked in the trend chart and/or the waveform chart corresponding to the medical and nursing operation. Specifically, in the solution of displaying the trend chart and/or the waveform chart corresponding to the medical and nursing operation, the alarm condition corresponding to the target physiological sign parameter is determined; and in the trend chart and/or the waveform chart corresponding to the medical and nursing operation, the parameter values that meet the alarm condition are marked for prompting.

In an exemplary marking method, the parameter values that meet the alarm condition in the trend chart and/or the waveform chart are set in a display pattern different from that of other parameter values. For example, the line color of the parameter value that meets the alarm condition is set to a different color.

In another exemplary marking method: in the trend chart and/or the waveform chart corresponding to the medical and nursing operation, the parameter value that meets the alarm condition is determined; and if multiple parameter values meet the alarm condition, the maximum and minimum values of the multiple parameter values are marked for prompting in the trend chart and/or the waveform chart corresponding to the medical and nursing operation. It should be noted that the maximum and minimum values of the multiple parameter values can be the maximum and minimum values of multiple parameter values of consecutive collection time points. That is to say, when a certain segment of consecutive line in the trend chart and/or the waveform chart meets the alarm condition, the maximum and minimum values are searched for marking in the multiple parameter values represented by the consecutive line segment. Of course, when multiple consecutive line segments in the trend chart and/or the waveform chart meet the alarm condition, the maximum and minimum values are respectively searched for marking in each line segment.

A further exemplary marking method comprises determining an alarm type of the parameter value that meets the alarm condition. The alarm types includes a high alarm type and a low alarm type, where the high alarm type is used to indicate that the parameter value of the target physiological sign parameter is greater than the target parameter value by a level higher than the preset proportion value, and the low alarm type is used to indicate that the parameter value of the target physiological sign parameter is smaller than the target parameter value by a level smaller than the preset proportion value. The medical device determines an alarm prompt corresponding to the alarm type; and displaying the alarm prompt at an associated position of the parameter value that meets the alarm condition.

As mentioned above, the alarm condition includes two situations in which different alarm types are set for different situations, and different alarm prompts are respectively set for different alarm types. The alarm prompt may be in the form of characters, symbols, graphics, pictures, etc.

For example, as shown in FIGS. 2A-2E and FIGS. 2F-2I below, the parameter values that meet the alarm condition in the trend chart and/or the waveform chart are marked with upward and downward arrows. The upward arrow indicates that the difference value between the parameter value of the target physiological sign parameter and the target parameter value is higher than the preset proportion value of the target parameter value, and the difference value between the parameter value of the target physiological sign parameter and the target parameter value is lower than the preset proportion value of the target parameter value. Of course, the arrows are merely illustrative, and the form of another symbol or another mark may also be provided. It should be noted that, in addition to marking the parameter values corresponding to the latest medical and nursing operation, the parameter values that meet the alarm condition in the review trend chart can also be marked.

On the basis of such a marking method, the physiological sign parameter display method can further include: displaying the preset proportion value at the associated position of the alarm prompt so as to prompt the user that the reason for the alarm is related to the preset proportion value. The associated position may be a position near the alarm prompt. It should be noted that if there are multiple parameter values that causes an alarm, the preset proportion value can be displayed only at the associated location of the alarm prompt that is closest to the current time point.

For example, as shown in FIGS. 2A-2E and FIG. 13F below, the preset proportion value of 20% is displayed near an alarm prompt (indicated by the upward or downward arrow) closest to the current time point, and the alarm prompt and the preset proportional value are enclosed by a line box. At that time, the line box can be omitted or in other forms.

Further, on the basis of marking the parameter values, the method can further include: displaying preset detailed information associated with the parameter value in response to a triggering operation on a certain marked parameter value by the user. Specifically, the user can click on the parameter values marked in the trend chart and/or the waveform chart, and then the medical device displays the detailed information of the parameter values. The detailed information is preset, and can include: the numerical value of the parameter values, the collection time point, the specific situation of the corresponding medical and nursing operation, etc.

Further, on the basis of marking the parameter values, the method can further include: in the trend chart and/or the waveform chart corresponding to the medical and nursing operation, the target parameter values corresponding to the target physiological sign parameters are prompted, so that the user can visually obtain the target parameter value corresponding to each target physiological sign parameter. In an example, the target parameter values may be prompted by: displaying the target parameter value and/or a mark line corresponding to the target parameter value in the trend chart and/or the waveform chart corresponding to the medical and nursing operation. The mark line may also be referred to as a numerical value line. In addition, the specific numerical value of the target parameter value can be displayed near the mark line, and the specific numerical value can be displayed within a mark symbol.

As shown in FIG. 13F, a horizontal line is added to the heart rate trend chart, and the value 80 is shown at the right end of the horizontal line to indicate that the target parameter value of the target physiological sign parameter of the heart rate is 80. In addition, the value 80 can be contained in the mark symbol such as a flag and a bubble.

If the mark line corresponding to the target parameter value is displayed, a line that does not coincide with the mark line is displayed in the trend chart and/or the waveform chart corresponding to the medical and nursing operation; and a region formed by the line and the mark line is filled with a background color. Specifically, according to this solution, all the numerical values that are not the target parameter values in the trend chart and/or the waveform chart are marked in such a manner that the regions enclosed by these lines and the mark lines are filled with the background color. Of course, other marking methods can also be provided.

Further, the real-time parameter value of the target physiological sign parameter can be compared with the target parameter value to prompt the user of the relationship between the real-time monitoring situation of the target physiological sign parameter and the target expectation.

Therefore, on the basis of displaying the trend chart and/or the waveform chart corresponding to the medical and nursing operation, the physiological sign parameter display method may further include: obtaining the real-time parameter value of the target physiological sign parameter of the monitored object, obtaining the target parameter value corresponding to the target physiological sign parameter; and displaying the real-time parameter value and the target parameter value. Further, the real-time parameter value and the target parameter value can also be compared to obtain a comparison result, and the comparison result is prompted so that the user can visually obtain the relationship between the real-time parameter value and the target parameter value.

A prompt method of the comparison result includes determining a content prompt of the comparison result, and displaying the content prompt. The comparison result of the real-time parameter value and the target parameter value may include that the real-time parameter value is greater than, smaller than or equal to the target parameter value, so as to respectively determine the content prompt corresponding to each situation.

As shown in FIG. 13G, the real-time parameter value of the heart rate is 108, and the target parameter value is 78, so the real-time parameter value is greater than the target parameter value, which is prompted with an upward arrow. In the same way, the prompt of the blood pressure can be obtained. The real-time parameter value of the respiration rate is 15, and the target parameter value is 25, so the real-time parameter value is smaller than the target parameter value, which is prompted with a downward arrow. Of course, if the target parameter value is prompted in the illustration in the manner of bubble with value, the target parameter value below the real-time parameter value can be omitted.

In addition to prompting the relationship between the real-time parameter value and the target parameter value, the relationship between the real-time parameter value and the preset proportional value may also be prompted. In an embodiment, on the basis of displaying the trend chart and/or the waveform chart corresponding to the medical and nursing operation, the physiological sign parameter display method may further include:
obtaining the real-time parameter value of the target physiological sign parameter of the monitored object, and obtaining an alarm condition corresponding to the target physiological sign parameter; determining an alarm type according to the real-time parameter value and the alarm condition; and prompting the alarm type. The alarm type can refer to the above description regarding the high alarm type and the low alarm type.

In an embodiment, a prompting method includes determining a content prompt of the alarm type, and displaying the content prompt. In the same way, different content prompts may be determined according to different situations where the real-time parameter value is greater than, smaller than, or equal to the target parameter value. The content prompt may be an arrow, a background color, etc.

As shown in FIG. 13H, the real-time parameter value of the heart rate is 108, the target parameter value is 78, and the preset proportion value is 20%, so a current proportion (108−78)/78 is greater than 20%, indicating a high alarm type, which is thus prompted with an upward arrow. In the same way, the prompt of the blood pressure can be obtained. The real-time parameter value of the respiration rate is 15, the target parameter value is 25, and the preset proportion value is 20%, so a current proportion (25−15)/25 is greater than 20%, indicating a low alarm type, which is thus prompted with a downward arrow. Of course, if the target parameter value is prompted in the illustration in the manner of bubble with value, the target parameter value below the real-time parameter value can be omitted. It should be noted that the preset proportion value may or may not be filled with the background color.

Alternatively, the real-time parameter value can be displayed in a rectangular region. If the real-time parameter value meets the alarm condition of the high alarm type, a triangle is displayed above the rectangular region, and the preset proportion value and the upward arrow corresponding to the high alarm type are displayed in the triangle. If the real-time parameter value meets the alarm condition of the low alarm type, another triangle is displayed under the rectangular region, and the preset proportion value and the downward arrow corresponding to the low alarm type are displayed in the triangle. Of course, in the triangle, the preset proportional value can be omitted, or the arrow corresponding to the alarm type can be omitted.

As shown in FIG. 13I, the real-time parameter value of the heart rate is 108, the target parameter value is 78, and the preset proportion value is 20%, so a current proportion (108−78)/78 is greater than 20%, indicating a high alarm type, such that the value 108 is displayed in the rectangular region, and a triangle is displayed above the rectangular region, with the preset proportion of 20% and an upward arrow being contained therein. In the same way, the relevant prompts of the blood pressure and the respiration rate can be obtained.

It should be noted that triangles with low brightness may be provided above and below the rectangular region, with the upper triangle corresponding to the high alarm type, and the lower triangle corresponding to the low alarm type. After the alarm type corresponding to the real-time parameter value is determined, the triangle corresponding to the alarm type is highlighted.

As mentioned above, the medical and nursing operation can be a medication related operation. In this case, in addition to displaying the parameter information of the target physiological sign parameter, the medication operation related information may also be displayed. Therefore, the physiological sign parameter display method may further include:
obtaining the medication operation related information that may include: one or more of the occurrence time of medication operation, a drug name, a total drug volume, the drug flow rate, and the used drug volume; and displaying the medication operation related information. In some embodiments, the physiological sign parameter interface contains the medication operation related information in addition to the parameter information, and the two kinds of information can be respectively displayed in different regions.

The medication related operation may include regulating the flow rate of the drug used. The medication operation related information can be displayed by generating a line chart including the flow rate before regulation and the flow rate after regulation according to the regulation of the flow rate of the drug used; and displaying the line chart. The line chart may be a horizontal line, which rises by one step when the flow rate of the drug is increased, whereas falls by one step when the flow rate of the drug is decreased. As shown in FIGS. 2A-2I, the drugs “dopamine”, “Yuelixi (midazolam injection)” and “norepinephrine” were respectively regulated in rate twice. In order to indicate the specific situation of the flow rate, flow rate values before and after the flow rate regulation can be marked, and in order to prompt the time point of the flow rate regulation, the time point of the flow rate regulation can be displayed at a line position corresponding to each time point of flow rate regulation.

If there are multiple drugs corresponding to the medication operation, the respective used drug volume of each drugs can be displayed. Alternatively, if there are multiple drugs corresponding to the medication operation, the total volume of the used drug volumes of all the drugs is displayed. Referring to FIG. 13B, the liquid infusion volume corresponds to the value 890, indicating the total used volume of the drugs “dopamine”, “midazolam injection” and “norepinephrine”.

Alternatively, when the total volume of the used drug volumes is displayed, the total volume can also be counted in time units. For example, the drug volume per hour from the start of medication to the current time point can be displayed. Referring to FIGS. 2A-2I, in the statistical histograms, the numerical values above the histograms represent the total volume of the used drugs per hour.

It should be noted that the events that occurs within 1 hour and the time point at which each event occurs are recorded in FIGS. 2A and 2C-2I, specifically including excessively low heart rate, excessively high heart rate, excessively high systolic pressure (Sys), excessively low systolic pressure (Sys), and bigeminal premature ventricular contraction.

As can be seen on the basis of the above illustrated example, a display method of the physiological sign parameter interface is as shown in FIG. 13J, including a title bar, a trend chart region, a medication information region, a liquid volume analysis region, an event overview region, and a setup region. Of course, it is also possible to cancel the display of some regions or add the display of other regions according to actual requirements. For example, as described above, the physiological sign parameter interface may further include the pump status region and/or the alarm and prompt region.

Moreover, it should be noted that the physiological sign parameter interface may be contained in the entire display interface, such as the display interfaces as shown in FIGS. 2A-2J, and the region filled with diagonal lines outside the physiological sign parameter interface can contain other information that needs to be monitored.

In practical applications, the medication operation related information can be displayed in a preset medication region, but this region may be too small to display all the related information, and the information can be displayed in split-view. Specifically, the preset medication region is determined in the physiological sign parameter interface, and the number of the drug related information that can be displayed in the preset medication region is set as a preset number. When the number of the drug related information corresponding to the medication operation is greater than the preset number, the preset number of the drug related information are displayed in the preset medication region; and in response to a switching operation by the user, the remaining number of the drug related information that are not displayed before the switching are displayed in the preset medication region.

The switching operation may be a swiping operation, for example, swiping upward, downward, to the left, or to the right to view the related information displayed in other split screens.

In the same way, the displayed parameter information may also be displayed in split-view. Specifically, a parameter information region is determined in the physiological sign parameter interface, and the number of the target physiological sign parameters that can be displayed in the parameter information region is provided with a preset number. When the number of the target physiological sign parameters is greater than the preset number, the preset number of the parameter information of the target physiological sign parameters are displayed in the parameter information region; and in response to the switching operation by the user, the remaining number of the parameter information of the target physiological sign parameter that are not displayed before the switching are displayed in the parameter information region.

As mentioned above, the parameter information can be displayed by means of the trend chart and/or the waveform chart. When there are many target physiological sign parameters, the trend charts and/or the waveform charts of some of the target physiological sign parameters are first displayed in the trend chart region, and the trend charts and/or the waveform charts displayed in other split screens are displayed on the basis of a switching operation, such as a swiping operation, by the user.

In addition, the disclosure further provides a medical device, comprising a display, a memory and a processor. In the medical device,
the display is configured to display information;
the memory stores executable program instructions; and
the processor executes the executable program instructions to implement the steps of any one of the physiological sign parameter display methods mentioned above.

A specific example of the medical device is a monitor, and an exemplary structure of the monitor is shown in FIG. 14. FIG. 14 provides a system framework diagram of a parameter processing module in a multi-parameter monitor.

The multi-parameter monitor has an independent housing, and a sensor interface region is arranged on a housing panel. multiple sensor interfaces are integrated in the sensor interface region and configured to be connected to various external physiological sign parameter sensor accessories 311. The housing panel further comprises a small IXD display region, a display 318, an input interface circuit 320, an alarm circuit 319 (such as an LED alarm region), and the like. The parameter processing module is used as an external communication and power source interface for communicating with a main unit and taking power from the main unit. The parameter processing module also supports a build-out parameter module, can form a plug-in monitor main unit by means of inserting the parameter module, can be used as part of the monitor, or can be connected to the main unit via a cable, with the build-out parameter module being used as an external accessory of the monitor. In addition, the multi-parameter monitor comprises a memory 317 for storing computer programs and various data generated during the related monitoring process.

An internal circuit of the parameter processing module is disposed in the housing, as shown in FIG. 14, and comprises signal collection circuits 312 corresponding to at least two physiological sign parameters, a front-end signal processing circuit 313, and a main processor 315.

The main processor 315 can implement the steps related to processing in each apnea event monitoring method mentioned above.

The signal collection circuits 312 can be selected from an electrocardiogram circuit, a respiration circuit, a body temperature circuit, a blood oxygen circuit, a non-invasive blood pressure circuit, an invasive blood pressure circuit, etc. The signal collection circuits 312 are respectively electrically connected to the corresponding sensor interfaces and are used to be electrically connected to the sensor accessories 311 corresponding to different physiological sign parameters, with an output end thereof being coupled to the front-end signal processor. A communication port of the front-end signal processor is coupled to the main processor, and the main processor is electrically connected to the external communication and power source interface.

Various physiological sign parameter measurement circuits can use common circuits in the prior art. The front-end signal processor completes sampling and analog-to-digital conversion of an output signal of the signal collection circuit, and outputs a control signal to control a measurement process of the physiological signal. These parameters include but are not limited to: parameters such as electrocardiogram, respiration, body temperature, blood oxygen, non-invasive blood pressure, and invasive blood pressure.

The front-end signal processor can be implemented by a single-chip microcomputer or other semiconductor devices, and can also be implemented by an ASIC or an FPGA. The front-end signal processor can be powered by an isolated power source. The sampled data is simply processed and packaged, and then sent to the main processor by means of the isolated communication interface. For example, the front-end signal processor circuit can be coupled to the main processor 315 by means of an isolated power source and communication interface 314.

The reason that the front-end signal processor is powered by an isolated power source is that the DC/DC power source isolated by a transformer plays a role in isolating the monitored object from a power supply device, with the main purposes including: 1. isolating the monitored object, and enabling an application part to be floating by means of the isolation transformer, so that a leakage current of the monitored object is small enough; and 2. preventing the voltage or energy during defibrillation or electrotome application from affecting a board card and a device of an intermediate circuit such as a main control board (guaranteed by a creepage distance and an electrical clearance).

The main processor completes the calculation of physiological sign parameters, and sends calculation results and waveforms of the parameters to the host (such as a host with a display, a PC, a central station, etc.) by means of the external communication and power source interface. The external communication and power source interface 316 may be one or a combination of local area network interfaces composed of Ethernet, a token ring, a token bus, and a fiber distributed data interface (FDDI) as the backbone of these three networks, may also be one or a combination of wireless interfaces such as an infrared interface, a Bluetooth interface, wife, WMTS communication, etc., or may also be one or a combination of wired data connection interfaces such as an RS232, a USB, etc.

The external communication and power source interface 316 may also be one or a combination of a wireless data transmission interface and a wired data transmission interface. The host may be any computer device such as the host of the monitor, an electrocardiograph, an ultrasonic diagnosis instrument, a computer, etc., and a monitoring device can be formed by means of installing with matching software. The main unit may also be a communication device such as a mobile phone, and the parameter processing module sends, by using a Bluetooth interface, data to the mobile phone supporting Bluetooth communication, so as to implement remote transmission of the data.

In addition, the disclosure provides a readable storage medium with a computer program stored therein, and when the computer program is executed by a processor, the above physiological sign parameter display method is implemented.

It should be noted that the embodiments in the specification are described in a progressive manner, and each of the embodiments focuses on differences from other embodiments, and the same or similar parts of the embodiments can be referred to each other.

It should also be noted that the terms of relationship herein, such as first and second, are used only to distinguish one entity or operation from another entity or operation, without necessarily requiring or implying any such actual relationship or sequence between these entities or operations. Moreover, the terms “comprise”, “include” or any variation thereof are intended to cover a non-exclusive inclusion, so that a process, method, article or device that comprises a series of elements not only comprises those elements but also comprises other elements not expressly listed or further comprises elements inherent to such a process, method, article, or device. In the absence of more restrictions, the element defined by the phrase “comprising a/an . . . ” does not exclude the presence of a further identical element in the process, method, article or device that comprises the element.

The above embodiments do not constitute a limitation on the scope of protection of the technical solutions. Any modification, equivalent replacement, improvement, etc. made within the spirit and principle of the above embodiments shall fall within the scope of protection of the technical solutions.

What is claimed is:

1. A monitor, comprising:
a physiological data monitoring apparatus;
a processor;
a display; and
a communication unit,
wherein the physiological data monitoring apparatus is configured to monitor one or more physiological sign parameters of a monitored object, the communication unit is configured to receive medication information from an infusion pump, and the processor is configured to control the display to display the one or more physiological sign parameters and the medication information in association.

2. The monitor of claim 1, wherein the medication information comprises at least one of a flow rate of a drug, a dose, a concentration, a cumulative infusion liquid volume, and a physiological sign parameter that can be affected during the use of the drug, and the physiological sign parameter that can be affected during the use of the drug is at least one of the one or more monitored physiological sign parameters.

3. The monitor of claim 1, wherein the processor is further configured to control to generate prompt information when unusual fluctuation occurs in any physiological sign parameter monitored by the physiological data monitoring apparatus.

4. The monitor of claim 3, further comprising a storage unit, wherein the storage unit stores a predefined correspondence between a fluctuation range of each physiological sign parameter and a prompt level, and the processor determines and controls to generate prompt information corresponding to the prompt level according to the correspondence and a current range of unusual fluctuation of the physiological sign parameter.

5. The monitor of claim 4, wherein the storage unit further stores a predefined duration threshold for triggering generation of a prompt information by unusual fluctuation of each physiological sign parameter, and the processor controls to generate prompt information when a duration of unusual fluctuation of any physiological sign parameter reaches a corresponding duration threshold.

6. The monitor of claim 3, wherein the prompt information comprises a combination of one or more of a pattern, color, text, voice, and video.

7. The monitor of claim 3, wherein the processor is further configured to, when it is determined, according to unusual fluctuation of a physiological sign parameter, that a current flow rate of the drug needs to be controlled, generate a drug flow rate control instruction, and control the communication unit to send the drug flow rate control instruction to the infusion pump, so that the infusion pump controls the flow rate of the drug of the infusion pump in response to the drug flow rate control instruction.

8. The monitor of claim 7, wherein the processor is further configured to, when it is determined, according to unusual fluctuation of a physiological sign parameter, that infusion needs to be started or stopped currently, generate an infusion start or stop instruction, and control the communication unit to send the infusion start or stop instruction to the infusion pump, so that the infusion pump starts or stops the infusion in response to the infusion start or stop instruction.

9. The monitor of claim 2, wherein when at least one of the flow rate of the drug, the dose, or the concentration changes or a new drug is added, the processor controls to generate a corresponding parameter curve or a mathematical expression of parameter value fluctuation, and controls the display to display the parameter curve or the mathematical expression of parameter value fluctuation.

10. The monitor of claim 9, wherein when it is determined that the flow rate of the drug increases, the processor controls the display to display a watch window, in which a change trend chart of a physiological sign parameter closely related to the drug changing following the flow rate of the drug within a preset time period is displayed with a current flow rate value of the drug highlighted.

11. The monitor of claim 10, wherein the processor is further configured to close the watch window in response to a close operation of a user, or the processor controls to close the watch window when the flow rate of the drug no longer changes within a preset time period.

12. The monitor of claim 1, wherein to control the display to display the one or more physiological sign parameters and the medication information in association, the processor is further configured to:
associate the one or more physiological sign parameters with the medication information in time; and
control the display to display a correlation between the one or more physiological sign parameters and a medication flow rate trend, a dose trend or a concentration trend over time.

13. The monitor of claim 12, wherein the processor is further configured to generate a trend chart or a waveform chart corresponding to the medication information according to the one or more physiological sign parameters associated with the medication information.

14. The monitor of claim 13, wherein the processor is configured to switch the review trend chart or the review waveform chart displayed currently to the trend chart or the waveform chart corresponding to the medication information.

15. The monitor of claim 13, wherein the processor is configured to display the trend chart or the waveform chart corresponding to the medication information concurrently with displaying the review trend chart or the review waveform chart.

16. The monitor of claim 15, wherein the processor is configured to mark the trend chart or the waveform chart corresponding to the medication information on the displayed review trend chart or the review waveform chart; or,
the processor is configured to divide a trend chart region in a physiological sign parameter interface into a first sub-region and a second sub-region according to a preset region proportion; display the review trend chart or the review waveform chart in the first sub-region; and display the trend chart or the waveform chart corresponding to the medical and nursing operation in the second sub-region; or,
the processor is configured to use two vertically overlapped layers in a trend chart region of a physiological sign parameter interface to respectively display the review trend chart or the review waveform chart and the trend chart or the waveform chart corresponding to the medication information.

17. The monitor of claim 1, wherein the processor being configured to control the display to perform associated display of the physiological sign parameters and the medication information comprises: associating one or more physiological sign parameters within a preset time period before medication with one or more physiological sign parameters within a preset time period after the medication, and then controlling the display to display an association between the physiological sign parameters and the use of the drug.

18. The monitor of claim 12, wherein to control the display to display the one or more physiological sign parameters and the medication information in association, the processor is further configured to:
control to present the one or more physiological sign parameters by a max-min-average trend chart,
wherein the max-min-average trend chart is formed by: obtaining maximum values, minimum values, and average values in a plurality of values of the one or more physiological sign parameters measured in each unit time within the preset time period, and then connecting all maximum values, all minimum values, and all average values within the preset time period to form respective curves, so as to form the max-min-average trend chart representing a physiological sign parameter trend, wherein the preset time period comprises a number of unit times.

19. The monitor of claim 1, wherein the processor is further configured to collect statistics on infusion amounts of different drugs infused into a body of the monitored object in each time period to obtain cumulative infusion amounts of different drugs in different time periods.

20. The monitor of claim 1, wherein the processor is further configured to collect statistics on types of abnormality events, the number of occurrences of the abnormality events, and key indicators in the abnormality events within a preset time period, and control the display to display the statistics.
